(19) United States
(12) Patent Application Publication
(10) Pub. No.: US 2026/0260761 A1
(43) Pub. Date: Sep. 3, 2026

(54) CARDIOVASCULAR RISK PREDICTION USING MULTIPLE INSTANCE LEARNING AND LESION-AWARE MODELS

(71) Applicant: Heartflow, Inc., Mountain View, CA (US)

(72) Inventors: Michiel SCHAAP, Oegstgeest (NL); Matthew SINCLAIR, London (GB); Adam UPDEPAC, Alamo, CA (US); Nan XIAO, San Jose, CA (US); Peter Kersten PETERSEN, Palo Alto, CA (US)

(21) Appl. No.: 19/552,016

(22) Filed: Feb. 27, 2026

Related U.S. Application Data
(60) Provisional application No. 63/764,988, filed on Feb. 28, 2025.
(60) Provisional application No. 63/974,715, filed on Feb. 3, 2026.

(51) Int. Cl. G16H 50/30; G16H 50/20

(52) U.S. Cl. CPC G16H 50/30; G16H 50/20

(57) ABSTRACT

A method of performing a cardiovascular risk assessment for a patient may comprise extracting a plurality of cardiovascular plaque, geometric, and functional significance parameters from raw patient-specific medical image data. The method may further comprise analyzing the plurality of cardiovascular plaque, geometric, and functional significance parameters using a risk score model to output patient-specific risk values. The risk score model may be configured to correlate the plurality of cardiovascular plaque, geometric, and functional significance parameters with predicted cardiovascular outcomes. The method may further comprise determining a likelihood of a major adverse cardiovascular event for the patient based on the patient-specific risk values.

### CROSS-REFERENCE TO RELATED APPLICATIONS

This application claims priority to U.S. Provisional Application 63/764,988, filed Feb. 28, 2025, and to U.S. Provisional Application 63/974,715, filed Feb. 3, 2026, the entire disclosures of which are hereby incorporated by reference in their entirety.

### FIELD OF INVENTION

The present disclosure relates to medical risk assessment systems using machine learning, and more particularly to cardiovascular risk prediction systems that employ neural networks to analyze lesion-specific features from medical imaging data for predicting major adverse cardiac events and providing diagnostic and treatment information regarding major adverse cardiac events.

### BACKGROUND

Cardiovascular disease remains a leading cause of morbidity and mortality worldwide, with major adverse cardiac events (MACE) representing significant clinical challenges in patient management. Traditional risk assessment methods often rely on clinical risk scores and basic imaging parameters, which may not capture the complex interplay of factors that contribute to cardiovascular disease and events. These conventional approaches may have limitations in accurately predicting patient-specific risks, particularly in cases where multiple cardiovascular parameters need to be considered simultaneously.

Medical imaging technologies, including computed tomography angiography, magnetic resonance imaging, and echocardiography, provide rich datasets of cardiovascular parameters that can be analyzed to assess patient risk. These imaging modalities can capture various anatomical and functional characteristics such as vessel morphology, plaque composition, myocardial perfusion, and cardiac function metrics. However, the manual interpretation of these diverse imaging parameters can be time-consuming and may be subject to inter-observer variability, potentially limiting the consistency and accuracy of risk assessments.

The epicardial adipose tissue (EAT) and pericoronary adipose tissue (PCAT) are fat deposits located around the heart. EAT is found on the surface of the heart, while PCAT is located around the coronary arteries. Both EAT and PCAT have been implicated in the development and progression of coronary artery disease (CAD), which is a leading cause of death worldwide. EAT and PCAT are metabolically active tissues that secrete a variety of substances, including hormones, cytokines, and adipokines. These substances can affect the function of the heart and blood vessels, and they may contribute to the development of CAD by promoting inflammation, oxidative stress, and insulin resistance. EAT and PCAT have also been shown to affect the stability of atherosclerotic plaques, which are fatty deposits that build up in the arteries. Unstable plaques are more likely to rupture and cause a heart attack.

EAT and PCAT can be characterized to a certain extent with Computed Tomography Angiography (CCTA). CCTA aims to quantify the attenuation of high-energy photons in the heart. The photons can have a certain user-defined energy level (often expressed in peak kilovoltage [kVp]) and turning the raw measurement data into a volumetric image is done through reconstruction algorithms that also have certain parameters. The units of the volumetric pixel elements (voxels) are defined in Hounsfield units (HU). These intensities are calibrated with the attenuation of air and water. Tube voltage (kVp) is a factor influencing the Hounsfield Unit (HU) values obtained in both EAT and PCAT measurements due to the energy dependence of X-ray attenuation. As a result, images acquired at different kVp values can yield different HU values for the same tissue, leading to potential inconsistencies in quantitative analysis.

Methodologies for characterizing EAT and PCAT may rely on a two-step process. The initial step may involve extracting specific features, often termed radiomics, from the volumetric image data. These features can encompass various metrics such as mean pixel intensity, variance, skewness, kurtosis, or texture measures within a defined region of interest. Subsequently, the extracted radiomic features may then be used as input for a machine learning algorithm trained to classify or characterize the EAT and PCAT based on these features. This two-step approach may suffer from a limitation: by relying on pre-defined, hand-crafted features, the process may inadvertently overlook subtle but patterns or relationships within the complex volumetric image data.

Machine learning approaches, particularly neural networks, have shown promise in analyzing complex medical datasets and identifying patterns that may not be readily apparent through traditional analysis methods. Neural networks can process multiple cardiovascular parameters simultaneously and may identify subtle relationships between imaging features and clinical outcomes to ascertain relative cardiovascular risk for a patient. By focusing on the extraction of rich information from local image features, particularly within areas indicative of disease, and combining this with patient-level clinical data, these models can achieve a more nuanced and accurate risk prediction. The application of these computational methods to cardiovascular risk prediction represents an opportunity to enhance the accuracy and efficiency of risk assessment by leveraging the comprehensive information available from modern imaging technologies.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computer-implemented method for cardiovascular risk prediction using a first risk model is provided. The method includes obtaining medical imaging data from a patient, the medical imaging data comprising coronary computed tomography angiography data. The method includes extracting lesion-specific features from the medical imaging data, wherein the lesion-specific features comprise plaque composition parameters, geometric parameters, and functional significance parameters for individual coronary lesions. The method includes processing the lesion-specific features through a neural network architecture comprising a lesion scoring network configured to generate individual lesion scores for each detected lesion. The method includes aggregating the individual lesion scores to generate an aggregated lesion score. The method includes combining the aggregated lesion score with non-lesion cardiovascular features and patient demographic features to generate combined feature data. The method includes processing the combined feature data through a risk prediction neural network to generate a cardiovascular risk score indicating probability of major adverse cardiac events.

According to other aspects of the present disclosure that utilize a second risk model, the method may include one or more of the following features. The lesion scoring network may comprise a multi-layer perceptron network. The lesion scoring network may comprise a transformer-based architecture with attention mechanisms for processing multiple lesions simultaneously. The aggregating may comprise summing the individual lesion scores. The aggregating may comprise using a Transformer Encoder to generate contextualized embeddings for lesion aggregation. The plaque composition parameters may comprise calcified plaque volume, non-calcified plaque volume, and plaque burden measurements. The geometric parameters may comprise diameter stenosis measurements, vessel curvature measurements, and positive remodeling indices. The functional significance parameters may comprise fractional flow reserve computed tomography values. The non-lesion cardiovascular features may comprise epicardial adipose tissue volume, epicardial adipose tissue burden, left ventricular myocardial mass, pericoronary adipose tissue measurements, features derived from cardiac ventricular and atrial volumes and left atrial appendage morphology, aortic plaque, carotid plaque, and cerebrovascular plaque. Additional patient-level features may comprise risk modifiers such as hypertension, diabetes, hyperlipidemia, age, sex, current medications, or other demographic information. Presence of high-risk plaque features including positive remodeling, low attenuation (low density), napkin-ring sign, and spotty calcification may also be included, either at the lesion level, patient level, or both. The risk prediction neural network may be trained with loss functions associated with time-to-event modeling. The cardiovascular risk score may be calibrated to predict risk over a specified time period. The method may include generating risk stratification categories based on the cardiovascular risk score.

According to another aspect of the present disclosure, a system for cardiovascular risk assessment using a third risk model is provided. The system includes a processor and a memory storing instructions that, when executed by the processor, cause the system to obtain coronary imaging data from a patient. The system extracts multiple lesion features from the coronary imaging data, wherein each lesion feature set corresponds to an individual coronary lesion. The system processes each lesion feature set through a shared lesion scoring neural network to generate lesion-specific risk scores. The system aggregates the lesion-specific risk scores using a neural aggregation mechanism. The system combines the aggregated lesion scores with patient-level cardiovascular parameters and demographic data. The system generates a patient-specific cardiovascular risk prediction using a survival analysis neural network.

According to other aspects of the present disclosure, the system may include one or more of the following features. The shared lesion scoring neural network may comprise multiple fully connected layers with activation functions. The neural aggregation mechanism may comprise summation of lesion scores. The neural aggregation mechanism may comprise a transformer architecture with self-attention mechanisms. The patient-level cardiovascular parameters may comprise whole-heart geometric measurements, adipose tissue quantifications, and hemodynamic parameters, Additional patient-level features may comprise risk modifiers such as hypertension, diabetes, hyperlipidemia, age, sex, current medications, or other demographic information. The survival analysis neural network may implement survival analysis modeling with neural network components. The system may generate calibration plots for validating risk prediction accuracy. The system may output risk scores as continuous probability values between zero and one. The system may categorize patients into discrete risk groups based on threshold values applied to the risk scores.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a medical diagnostic interface displaying patient data categories and survival curve analysis for cardiovascular risk assessment, according to aspects of the present disclosure.

FIG. 2 depicts a neural network architecture for lesion-aware risk scoring using MLP lesion scoring and simple aggregation, according to aspects of the present disclosure.

FIG. 3 shows a transformer-based lesion score architecture that processes multiple lesion inputs through a Transformer Encoder, according to aspects of the present disclosure.

FIG. 4 illustrates a neural network architecture for lesion-aware cardiovascular risk prediction that processes raw patch data from multiple lesions, according to aspects of the present disclosure.

FIG. 5 presents distribution plots of censoring and event times in training data, according to aspects of the present disclosure.

FIG. 6 displays training and validation metrics for an MLP lesion scorer with simple aggregation using training data, according to aspects of the present disclosure.

FIG. 7 shows training and validation metrics for a transformer-based lesion score model using training data, according to aspects of the present disclosure.

FIG. 8 depicts a calibration plot for 5-year cardiovascular risk assessment using training data and the first risk model, according to aspects of the present disclosure.

FIG. 9 depicts a calibration plot for 5-year cardiovascular risk assessment using training data and the second risk model, according to aspects of the present disclosure.

FIG. 10 illustrates a Brier score curve over time using training data and the first risk model, according to aspects of the present disclosure.

FIG. 11 shows a Brier score analysis over time using training data and the second risk model, according to aspects of the present disclosure.

FIG. 12 depicts cumulative dynamic AUC performance over time using training data and the first risk model, according to aspects of the present disclosure.

FIG. 13 presents cumulative dynamic AUC performance over time using training data and the second risk model, according to aspects of the present disclosure.

FIG. 14 shows a histogram of predicted risk score distribution using training data and the first risk model, according to aspects of the present disclosure.

FIG. 15 displays a histogram of predicted risk scores using training data and the second risk model, according to aspects of the present disclosure.

FIG. 16 illustrates grouped survival curves based on predicted 5-year risk using training data and the first risk model, according to aspects of the present disclosure.

FIG. 17 presents grouped survival curves for cardiovascular risk assessment using training data and the second risk model, according to aspects of the present disclosure.

FIG. 18 depicts a scatter plot showing the relationship between aggregated lesion score and predicted risk score using training data and the first risk model, according to aspects of the present disclosure.

FIG. 19 shows a scatter plot of lesion score versus predicted risk using training data and the second risk model, according to aspects of the present disclosure.

FIG. 20 presents distribution plots for training and validation datasets showing censoring and event times using actual data, according to aspects of the present disclosure.

FIG. 21 displays training and validation metrics for MLP lesion scorer with simple aggregation using actual data, according to aspects of the present disclosure.

FIG. 22 shows training and validation metrics for transformer-based lesion score model using actual data, according to aspects of the present disclosure.

FIG. 23 depicts a calibration plot for 5-year risk assessment using actual data and the first risk model, according to aspects of the present disclosure.

FIG. 24 presents a calibration plot showing predicted risk versus observed risk using actual data and the second risk model, according to aspects of the present disclosure.

FIG. 25 illustrates a Brier score curve over time using actual data and the first risk model, according to aspects of the present disclosure.

FIG. 26 shows Brier score evolution over time using actual data and the second risk model, according to aspects of the present disclosure.

FIG. 27 depicts cumulative dynamic AUC performance over time using actual data and the first risk model, according to aspects of the present disclosure.

FIG. 28 presents cumulative dynamic AUC performance over time using actual data and the second risk model, according to aspects of the present disclosure.

FIG. 29 shows a histogram of predicted risk scores using actual data and the first risk model, according to aspects of the present disclosure.

FIG. 30 displays predicted risk score distribution using actual data and the second risk model, according to aspects of the present disclosure.

FIG. 31 illustrates grouped survival curves based on predicted 5-year risk using actual data and the first risk model, according to aspects of the present disclosure.

FIG. 32 presents grouped survival curves based on predicted 5-year risk using actual data and the second risk model, according to aspects of the present disclosure.

FIG. 33 depicts a scatter plot showing the relationship between lesion score and predicted risk using actual data and the first risk model, according to aspects of the present disclosure.

FIG. 34 shows a scatter plot with lesion score versus predicted risk using actual data and the second risk model, according to aspects of the present disclosure.

FIG. 35 illustrates integrated C/D AUC performance comparison showing horizontal bars for different risk model configurations, according to aspects of the present disclosure.

FIG. 36 shows a Receiver Operating Characteristic curve comparing model performance for predicting culprit lesions using the first risk model, according to aspects of the present disclosure.

FIG. 37 presents a block diagram of lesion-aware risk prediction system showing the determination of culprit lesions during processing, according to aspects of the present disclosure.

FIG. 38 illustrates a transformer-based architecture for lesion scoring with hierarchical processing structure, according to aspects of the present disclosure.

FIG. 39 depicts a computer system or environment for performing the various techniques described herein.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

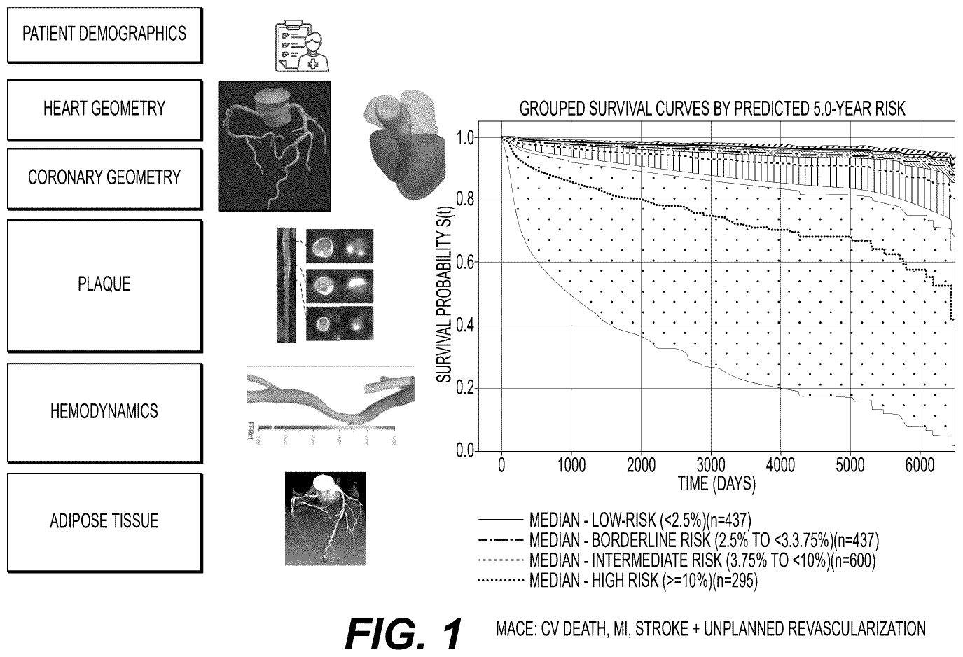

Referring to FIG. 1, a medical diagnostic interface may display comprehensive patient data categories and survival curve analysis for cardiovascular risk assessment. The interface may include inputs for patient demographics, heart geometry, coronary geometry, plaque characteristics, hemodynamics, and adipose tissue measurements, accompanied by relevant medical imaging visualizations. The survival curves may show probability on the y-axis ranging from 0.0 to 1.0 versus time in days on the x-axis for different risk groups based on predicted 5.0-year risk thresholds.

In some aspects, the risk categories may be defined by specific thresholds: low risk (less than 2.5%), borderline risk (2.5% to less than 3.75%), intermediate risk (3.75% to less than 10%), and high risk (greater than or equal to 10%). The survival curves may demonstrate varying trajectories for different risk groups, with shaded regions representing confidence intervals around the survival estimates. The interface may present MACE (major adverse cardiac events) outcomes, specifically tracking cardiovascular death, myocardial infarction, stroke, and/or unplanned revascularization, including outputting risk scores of these events and/or predicting time to these events.

In one technique, patient metadata, vessel, lesion, and other coronary data derived from imaging may be put into a survival regression to determine a risk score of MACE. However, simpler regressions may not easily account for interactions between multiple lesions, their spatial distribution, or morphological characteristics. Three-dimensional information may be lost, and nonlinear dependencies between lesions and clinical features may be lost. Techniques discussed herein may allow for more fine-grained, patient-specific lesion interactions to influence risk prediction, allowing for more accurate and personalized MACE risk predictions.

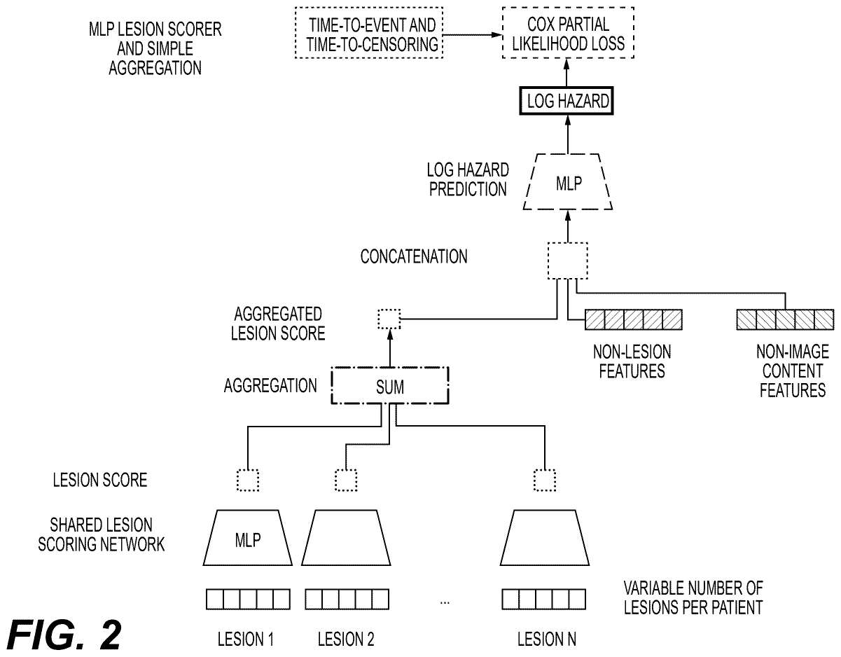

Referring to FIG. 2, a neural network architecture for lesion-aware risk scoring may employ MLP lesion scoring with simple aggregation mechanisms, referred to herein as a “first risk model.” The architecture may process multiple lesion inputs through a hierarchical structure, with first lesion input 1, second lesion input 2, and any number of additional lesion inputs N feeding into shared lesion scoring networks implemented as MLPs.

The lesion inputs may comprise one or more feature vectors in techniques discussed herein, wherein a vector may be generated for each lesion. The number of lesion vectors N may be automatically generated based on a determined number of lesions for the patient. The lesion-level features in the vector may comprise lesion plaque totals such as total calcified plaque volume, total non-calcified plaque volume, total plaque burden of lesion, calcified plaque burden, and non-calcified plaque burden. Plaque composition/morphology metrics may be included, such as non-calcified plaque volume fraction, low attenuation plaque volume fraction. Presence of high risk plaque features may be included, such as positive remodeling, low attenuation (low density), napkin-ring sign, and spotty calcification. Lesion-specific geometry metrics may be included, such as the percent diameter of stenosis (% DS) at the minimal lumen area (MLA), a positive remodeling index, a max curvature along the centerline, and a presence of bifurcation. Position features may be included, such as the percentage of myocardium affected. Functional significance information may be included, such as the imaging-derived fractional flow reserve (FFRct), delta FFRct, inflammation information such as PCAT (pericoronary adipose tissue) and/or EAT (epicardial adipose tissue) attenuation data, normalized mean PCAT attenuation, etc.

Both here and throughout this specification, MLPs may be substituted for other neural networks, including graph neural networks (GNN), which may preserve lesion relationships, a transformer/attention-based network, a 1D convolutional network (CNN), a recurrent neural network (RNN), a mixed density network or gaussian process layer, etc. Non-neural network techniques may also be used, such as gradient boosted trees, random forest algorithms, logistic regression or generalized linear models, kernel methods, etc.

The first lesion input 1, second lesion input 2, etc., may be vectors of features, each vector being associated with one lesion in a patient's vasculature, for example the coronary arteries. Each lesion vector may be provided to an MLP that determines a lesion score for each lesion. The lesion scores may be aggregated, for example, using a sum operation, to determine a combined/aggregated lesion score. Instead of a sum operation, other aggregations may be performed, such as maximum, minimum, median, weighted sum or weighted average aggregations. Learned/neural aggregators may be used, such as attention mechanism, set transformer/DeepSets aggregation, recurrent aggregation (RNN), graph-based pooling, and/or multi-head pooling. Distributional or statistical aggregation may be used, such as by using a histogram, percentile-based aggregation, top-k pooling, and/or entropy-weighted aggregation. Hierarchical or multi-vector aggregation may be used. A vector of aggregated features, such as max, min, variance concatenated, etc., may be used.

The system may combine the aggregated lesion score, for example through concatenation, with one or more non-lesion features and one or more non-image content features. The non-lesion features used throughout herein may include, e.g., non-lesion calcified plaque volume, non-lesion non-calcified plaque volume, left ventricular (LV) myocardial mass, volume to mass (V/M) ratio, volumes of cardiac ventricles and atria, epicardial adipose tissue (EAT) burden and/or volume, minimum CT-derived fractional flow reserve (FFRct), features derived from left atrial appendage morphology, aortic plaque, carotid plaque, and cerebrovascular plaque, etc. The non-image content features used herein may include age, sex, clinical risk factors, patient history, medications information, and other patient demographic and/or biographic information.

The lesion scores may include parameters relating to plaque volume, plaque type (e.g., calcified, non-calcified, low attenuation, etc.), and other relevant cardiovascular parameters, geometric parameters, derived parameters determined by combining or processing geometric, blood flow parameters, etc. The combination of the aggregated lesion score with non-lesion features and/or non-image content features discussed herein may be accomplished using elementwise addition, which adds the corresponding elements of each feature vector, elementwise multiplication, elementwise subtraction or division, averaging or weighted sum, and/or outer product. Learned (parametric) fusion mechanisms may be used such as attention-based fusion, which learns weights to focus on the most informative modality or feature type; gating or feature-wise linear modulation, where one feature vector may control the scaling and shifting of others; bilinear or tensor fusion layers, which learns interactions between modalities efficiently; cross-modal transformers, which model relationships between lesion-based and clinical features dynamically via attention; or learned projection and addition, which project both feature types into a shared latent space before summing or concatenating. Statistical or heuristic fusion techniques such as feature selection or weighting based on feature importance, for example by combining only top-ranked features or those above a threshold; rule-based or conditional fusion, for example combining lesion scores only if certain non-lesion features meet criteria (e.g., age >60); dimensionality reduction before fusion, such as autoencoder or learned embedding before combining. Hierarchical or multi-stage fusion may be used, such as combining lesion-derived and non-lesion features at multiple network layers rather than once.

The concatenated data may be provided to a MLP (multi-layer perceptron) that generates a log hazard prediction, comprising a scalar risk score for each patient, where the higher the log hazard prediction, the higher the instantaneous risk of the event occurring.

The log hazard output may be used to determine a survival analysis loss function, such as the Cox partial likelihood loss, as shown at the top of the diagram. Time-to-event and/or time-to-censoring may be needed during training to compute the survival loss values, while during production/inference, the underlying patient-specific relative risk estimation may be the output used to determine likely time to events for the patient, and the survival loss function might not be evaluated. The architecture may accommodate a variable number of lesions per patient and processes them through identical scoring networks before combining their information for final risk assessment via the first risk model.

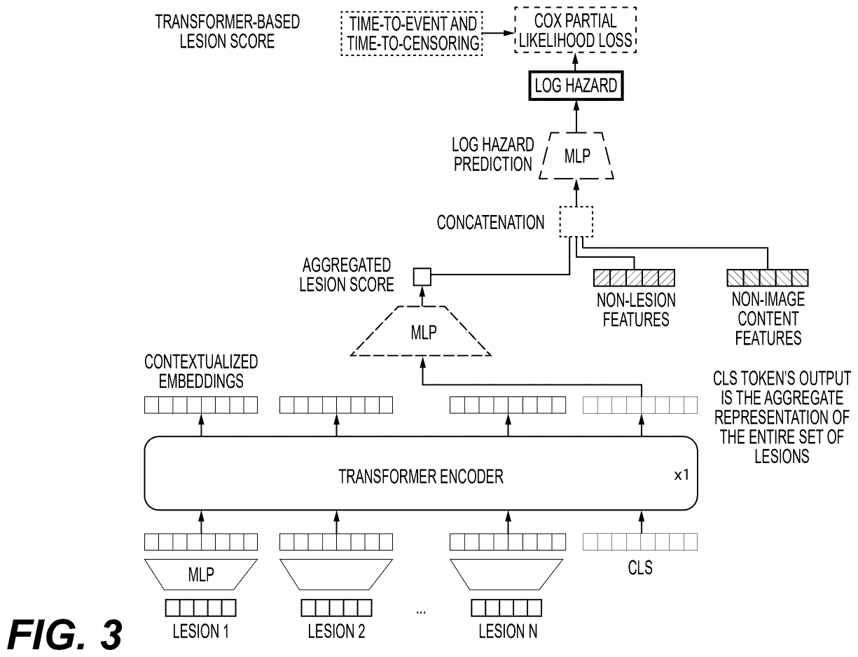

Referring to FIG. 3, a transformer-based lesion score architecture may process multiple lesion inputs through a Transformer Encoder, referred to herein as a “second risk model,” but which may incorporate features described above and elsewhere herein. The architecture may determine a feature vector for each lesion with first lesion input 1, second lesion input 2, and any number of additional lesion inputs N, each of which may be put through an MLP. In training, the MLP may learn a richer embedding per lesion, possibly normalizing features or projecting into a common latent space. The post-MLP embeddings may be put into a single Transformer Encoder. Additionally, a classification token (CLS) may also be provided to the Transformer Encoder. The Transformer Encoder may output contextualized embeddings that may be ignored in favor of the contextualized CLS vector in further processing. Alternatively, the contextualized embeddings may be provided to downstream tasks for lesion-specific predictions, such as lesion-level risk scoring or segmentation.

The Transformer Encoder may use self-attention, so every lesion embedding can interact with every other lesion embedding and with the CLS token. The CLS token may comprise the attention-weighted summary of all lesion embeddings plus the CLS original vector. Accordingly, the CLS token may represent the aggregate content of all lesions. The CLS token may further be provided to another MLP to form an aggregated lesion score.

The system may incorporate a concatenation layer that combines the contextualized embeddings with non-lesion features and non-CCTA features, such as those described herein in reference to the first risk model. The concatenated data may flow through an MLP layer that generates a log hazard output, which connects to a Cox partial likelihood loss component at the top of the diagram. The architecture may accommodate a variable number of lesions per patient and processes them before combining their information for final risk assessment.

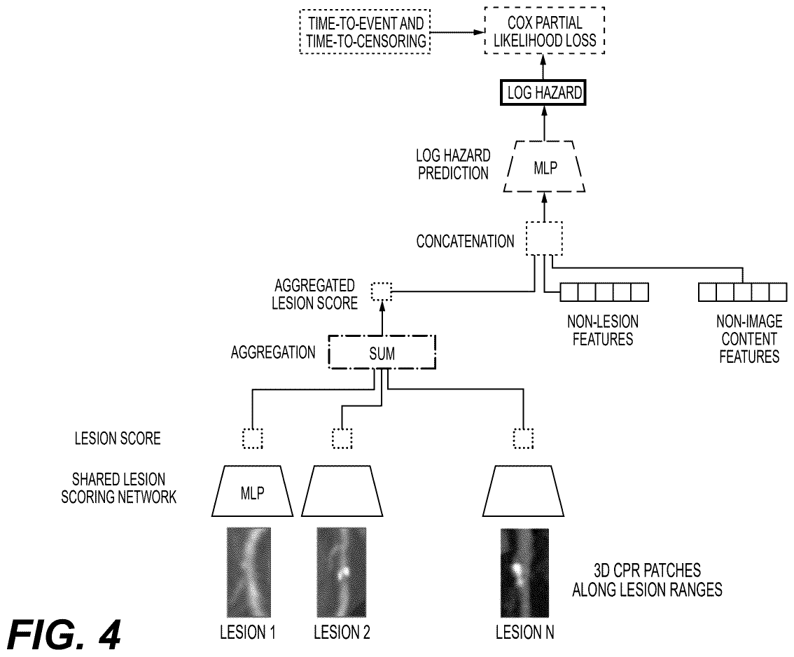

Referring to FIG. 4, a neural network architecture for lesion-aware/voxel-aware cardiovascular risk prediction may process raw 3D curved planar reformation (CPR) patch data from multiple lesions, or 3D image patches along the vessels, referred to herein as a “third risk model.” The system may receive multiple lesion inputs, for example three-dimensional sections of vessel that have been processed into a curved planar reformation or axis-aligned or vessel-aligned 3D image patches, and then trimmed into a separate patch for each lesion. A first lesion input 1, second lesion input 2, and any number of additional lesion inputs N may be received, which may be provided to MLPs, or other models discussed herein, to generate lesion-specific scores. The lesion scores may be aggregated using a sum or other operation to form an aggregated lesion score. The system may combine the aggregated lesion score through concatenation with non-lesion features and non-image content features described elsewhere herein.

The concatenated data may be provided to an MLP, or other model discussed herein, that determines a patient-specific relative risk estimation. This patient-specific relative risk prediction, as discussed herein, may be used to determine event probability over time and other risk metrics. In training, the patient-specific relative risk prediction may be provided to a survival loss determination, as discussed above. The architecture may accommodate a variable number of lesions per patient and processes them through scoring networks before combining their information for final risk assessment.

It is to be understood that any of the first, second, or third risk models may utilize the following formula (I) to categorize risk and generate a risk score report using Cox proportional hazards, here exampled with the mean-centered covariate form:

$$h ( t \vert X ) = {h}_{0} ( t ) \cdot exp ( {\sum }_{i = 1}^{p} {β}_{i} ( {X}_{i} - {\overline{X}}_{i} ) )$$

$$S ( t \vert X ) = {{S}_{0} ( t )}^{exp ( {\sum }_{i = 1}^{p} {β}_{i} ( {X}_{i} - {\overline{X}}_{i} ) )}$$

This formula (I) may utilize parameters such as age, sex, max DS, max main vessel curvature, occlusion, plaque total volume, calcified plaque volume, noncalcified plaque volume, plaque burden, minimum reportable FFRct, maximum delta FFRct, summed delta FFRct across lesions, epicardial adipose tissue volume, epicardial adipose tissue burden, and proximal RCA relative to pericoronary adipose tissue, etc., to determine a risk score and/or risk report for a patient.

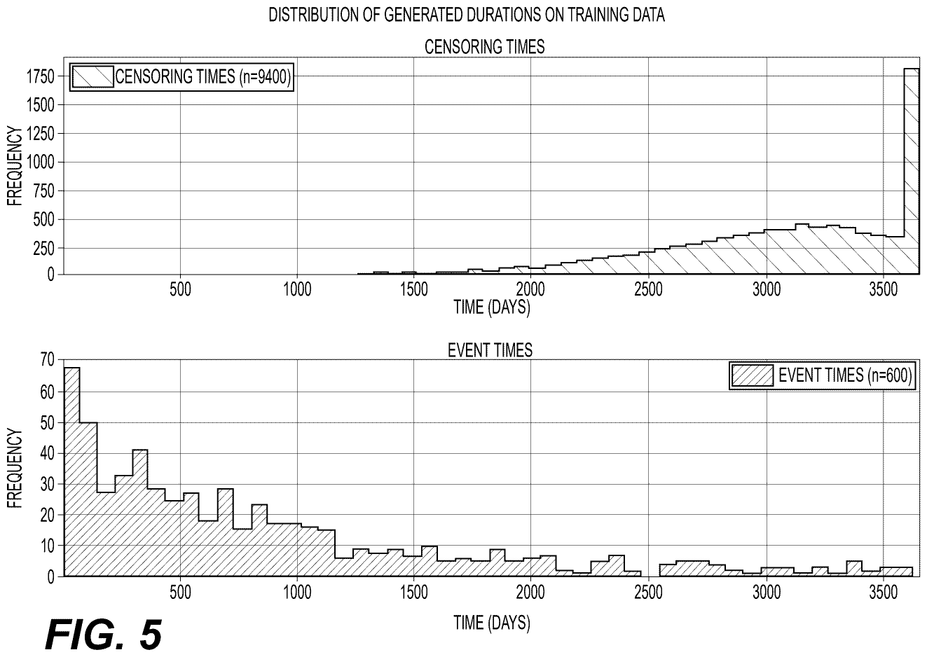

With continued reference to FIG. 5, distribution plots may show censoring and event times across training (e.g., synthetic) data with two distinct histograms. The upper graph may display the distribution of censoring times with a histogram showing frequencies ranging from 0 to approximately 1750. The lower graph may present the distribution of event times with a histogram showing frequencies ranging from 0 to approximately 70, demonstrating higher frequencies at earlier time points with a long tail extending toward later times.

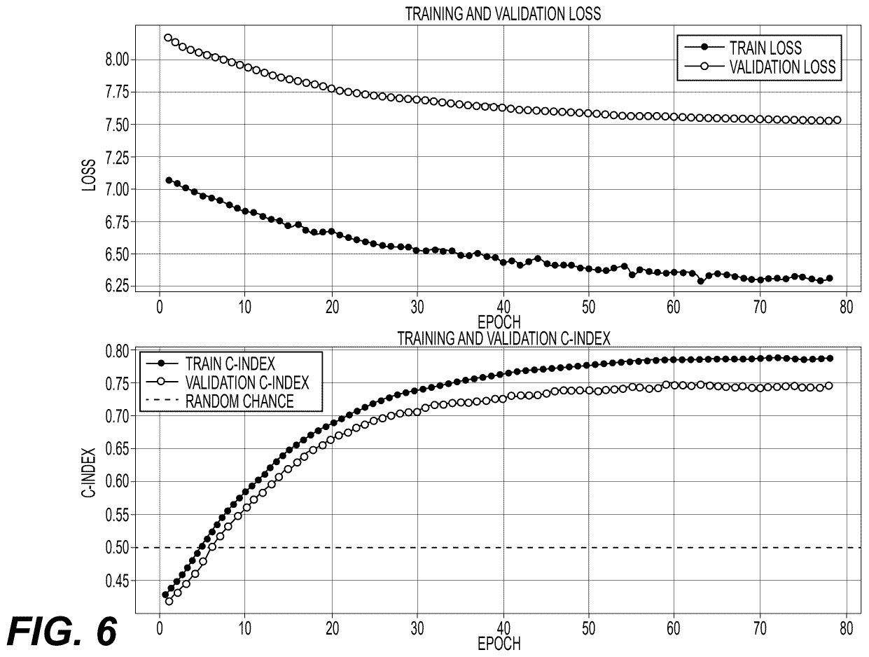

Referring to FIG. 6, training and validation metrics for an MLP lesion scorer with simple aggregation (e.g., the first risk model) may be displayed over approximately 80 epochs with two vertically arranged graphs. The upper graph may show Training and Validation Loss plotted against epochs, with the training loss curve starting at approximately 8.5 and rapidly decreasing before stabilizing around 5.5, while the validation loss curve begins near 7.0 and gradually decreases to stabilize around 6.5. The lower graph may display Training and Validation C-Index metrics plotted against epochs, with both curves showing improvement from initial values around 0.6 to plateau near 0.8. A dashed line indicating Random Chance performance may remain constant at 0.5 throughout the training period.

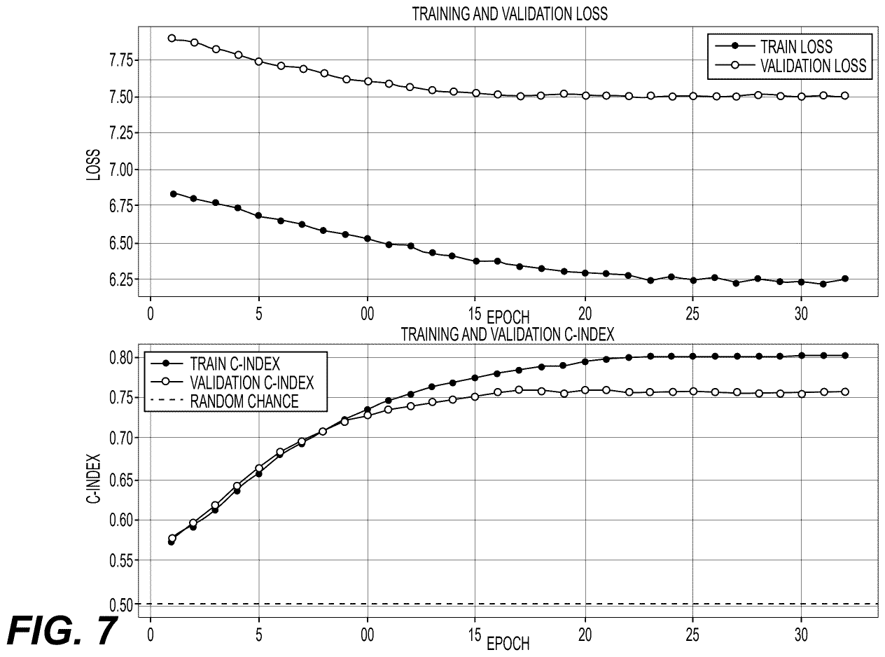

Referring to FIG. 7, training and validation metrics for a transformer-based lesion scorer (e.g., the second risk model) may be shown. The upper graph may show Training and Validation Loss with the Train Loss curve starting at approximately 6.8 and rapidly decreasing to stabilize around 6.25, and the Validation Loss curve beginning near 7.9 and gradually decreasing to stabilize around 7.5. The lower graph may display Training and Validation C-Index with both curves starting near 0.58 and increasing to plateau around 0.8. A dashed line labeled “Random Chance” may appear at 0.5 on the C-Index graph.

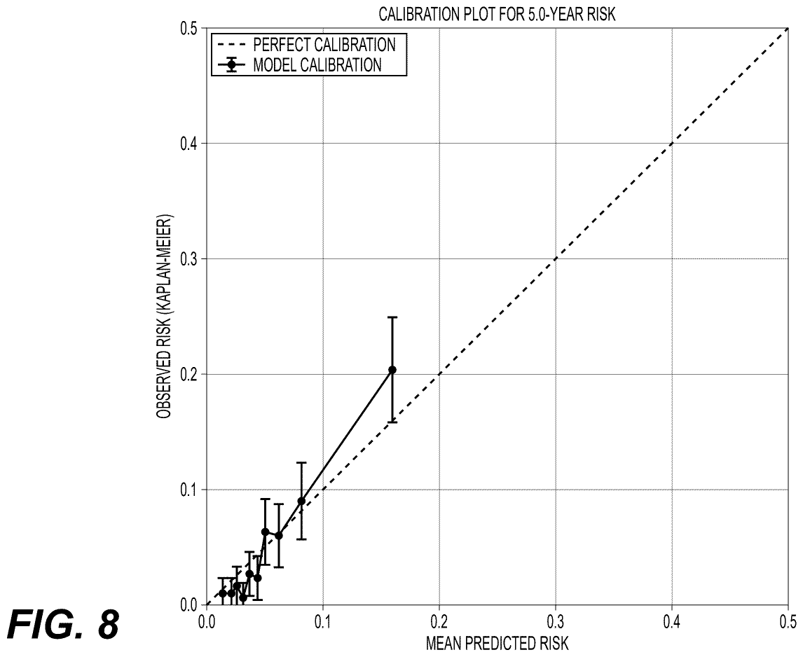

Referring to FIG. 8, a calibration plot for a 5.0-year cardiovascular risk assessment model may compare predicted risk with observed outcomes for an MLP lesion scorer with simple aggregation (e.g., the first risk model). The plot may display a dashed diagonal line representing perfect calibration and a line with error bars representing model calibration performance. The x-axis may show mean predicted risk ranging from 0.0 to 0.5, while the y-axis may show observed risk (Kaplan-Meier) also ranging from 0.0 to 0.5. The model calibration line may track close to the perfect calibration line at lower risk values below 0.1, with some deviation visible at higher risk levels around 0.2.

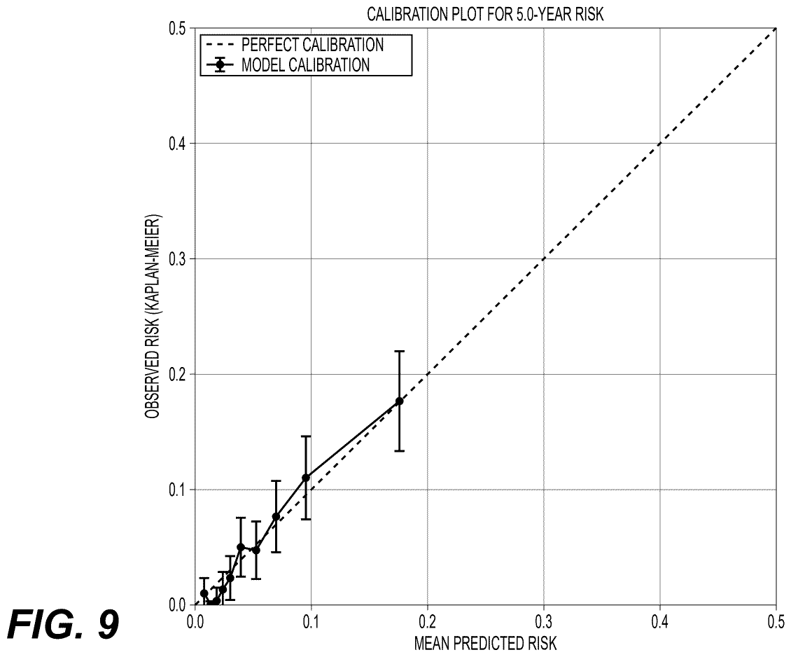

Referring to FIG. 9, another calibration plot for 5.0-year cardiovascular risk assessment may be presented with similar axes and layout to FIG. 8, but for a transformer-based lesion scorer (e.g., the second risk model). The plot may display a dashed diagonal line representing perfect calibration and a line with error bars representing model calibration performance. The model calibration line may track close to the perfect calibration line at lower risk values below 0.1, with some deviation above the perfect calibration line around 0.2.

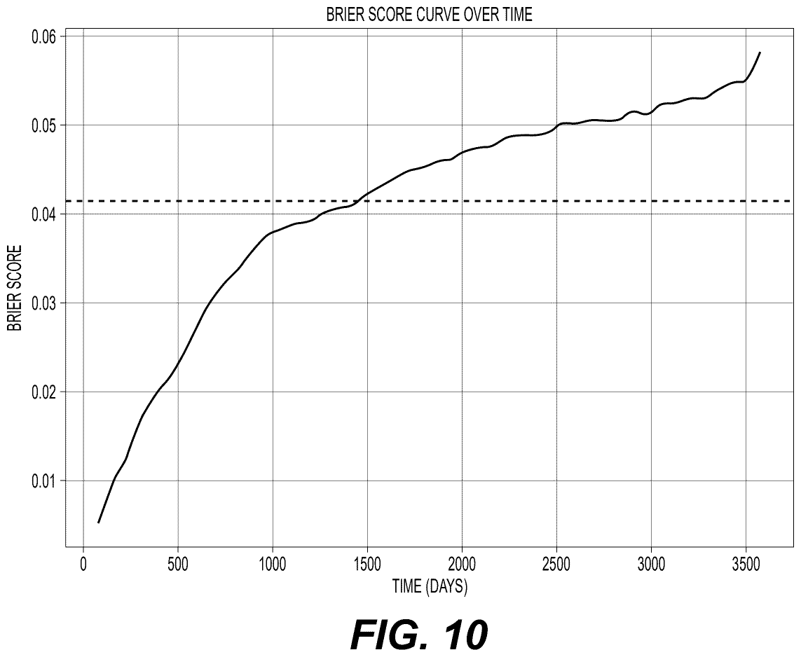

Referring to FIG. 10, a Brier score curve may be plotted over time with specific temporal patterns for an MLP lesion scorer with simple aggregation (e.g., the first risk model). The graph may display a curve representing Brier score values on the y-axis ranging from 0 to approximately 0.06, with a horizontal dashed reference line positioned at approximately 0.042. The Brier score curve may demonstrate an initial steep increase from near zero, intersecting the reference line around day 1500, and continues to rise more gradually thereafter, reaching approximately 0.058 by day 3500.

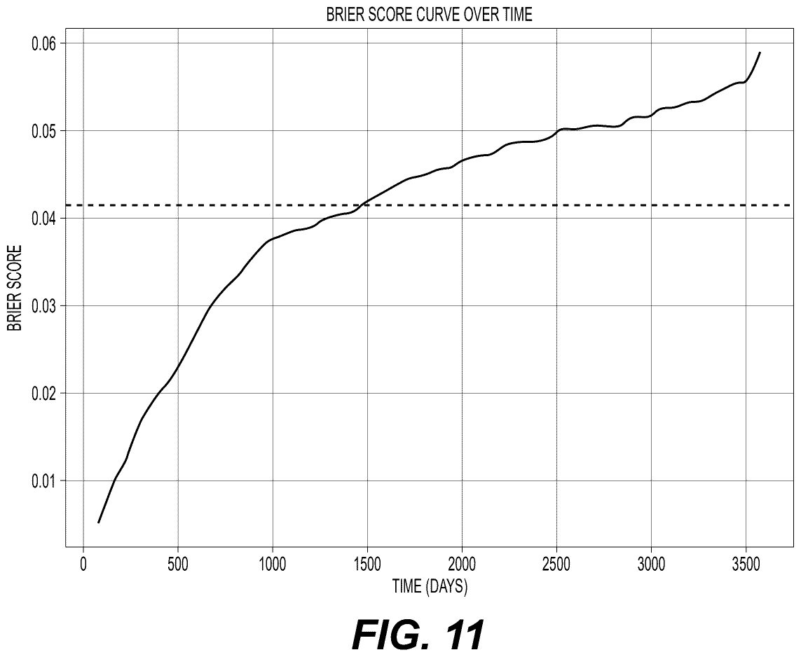

Referring to FIG. 11, another Brier score analysis may be shown with a similar pattern to FIG. 10, but for a transformer-based lesion scorer (e.g., the second risk model). The graph may display a curve representing Brier score values on the y-axis ranging from 0 to approximately 0.06, with a horizontal dashed reference line positioned at approximately 0.042. The Brier score curve may demonstrate an initial steep increase from near zero, intersecting the reference line around day 1500, and continues to rise more gradually thereafter.

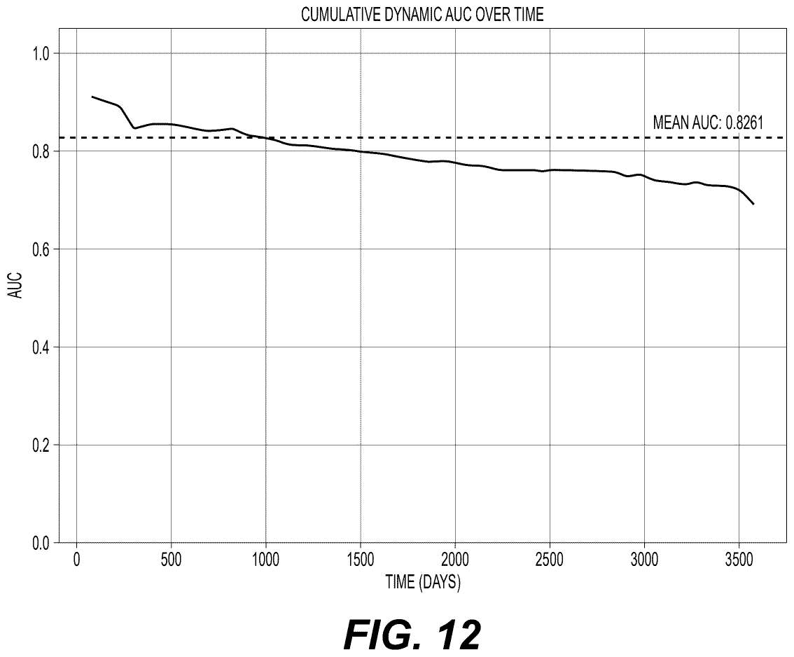

Referring to FIG. 12, cumulative dynamic AUC performance may be plotted over time with declining performance characteristics for an MLP lesion scorer with simple aggregation (e.g., the first risk model). The graph may display a curve representing dynamic Area Under Curve (AUC) values that starts at approximately 0.9 and gradually decreases over a period of 3500 days. A horizontal dashed line may indicate the Mean AUC value of 0.8261. The AUC curve may demonstrate some initial fluctuations in the early period before showing a general downward trend, maintaining values between 0.7 and 0.8 after day 1000.

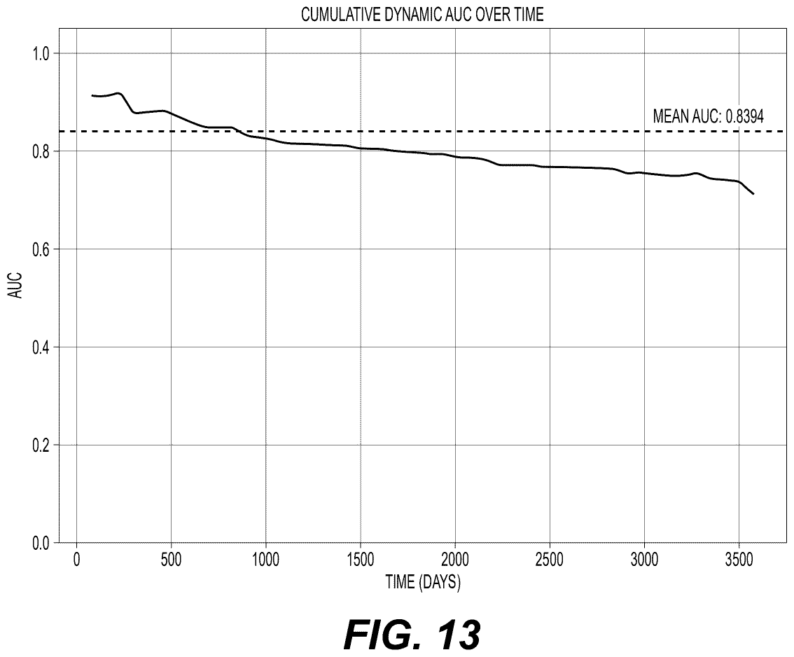

Referring to FIG. 13, cumulative dynamic AUC performance may be presented with similar characteristics to FIG. 12, but for a transformer-based lesion scorer (e.g., the second risk model). The graph may display a curve representing dynamic AUC values that starts at approximately 0.9 and gradually decreases over a period of 3500 days, with a horizontal dashed line indicating a mean AUC value of 0.8394. The AUC curve may demonstrate initial fluctuations in the early period before showing a general downward trend, eventually stabilizing between 0.7 and 0.8 after day 1000.

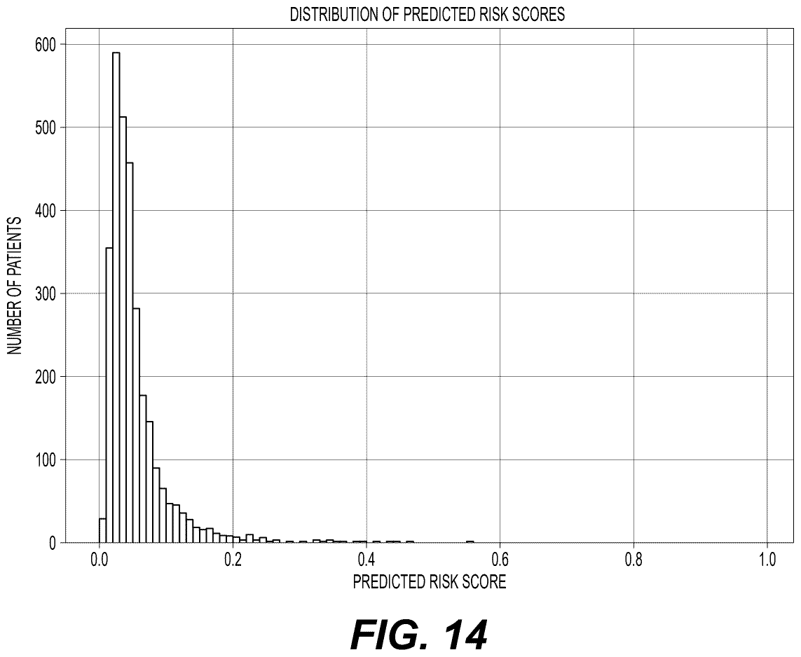

Referring to FIG. 14, a histogram may display the distribution of predicted risk scores across the patient population using an MLP lesion scorer with simple aggregation (e.g., the first risk model). The x-axis may represent predicted risk scores ranging from 0.0 to 1.0, while the y-axis may show the number of patients ranging from 0 to approximately 600. The distribution may exhibit a heavily right-skewed pattern, with the highest frequency occurring near zero risk scores and rapidly decreasing as risk scores increase. The majority of patients may have risk scores between 0.0 and 0.2, with very few patients having scores above 0.4.

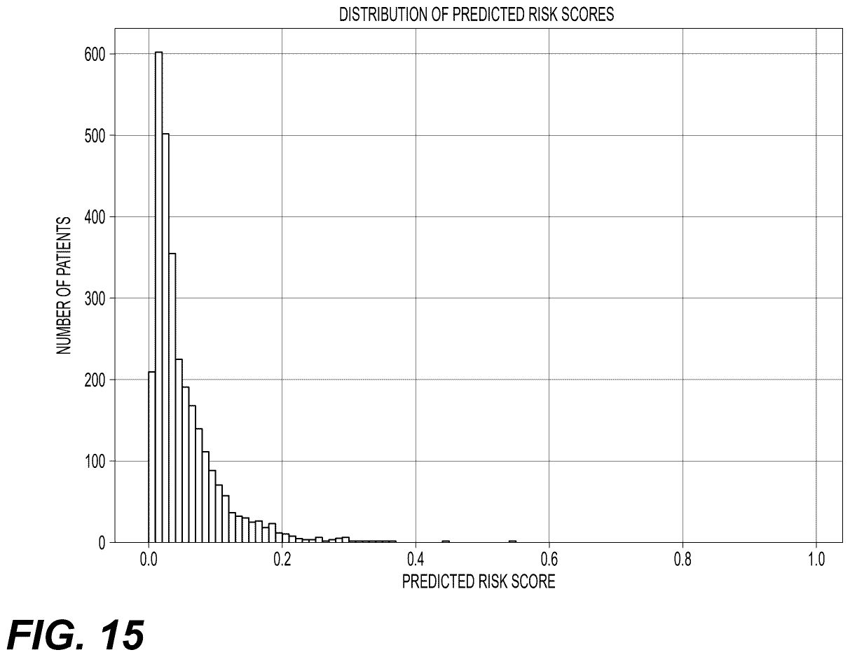

Referring to FIG. 15, another histogram may show predicted risk score distribution with similar characteristics to FIG. 14 but that utilizes a transformer-based lesion scorer (e.g., the second risk model). The distribution may exhibit a heavily right-skewed pattern, with the highest frequency occurring near zero risk scores and rapidly decreasing as risk scores increase. The majority of patients may have predicted risk scores below 0.2, with very few patients having scores above 0.4.

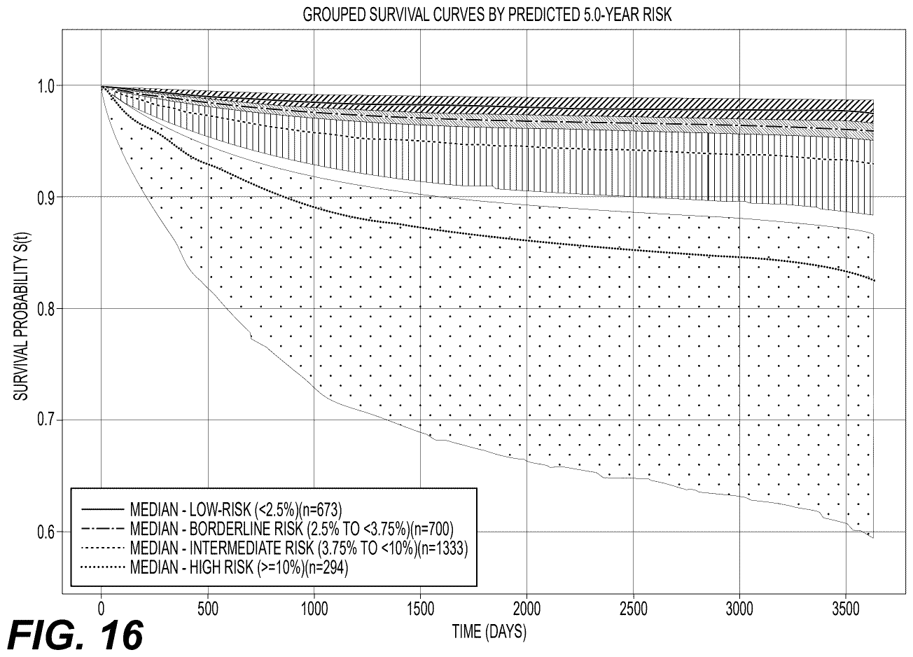

Referring to FIG. 16, grouped survival curves based on predicted 5.0-year risk assessment data may be illustrated with clear risk stratification for an MLP lesion scorer with simple aggregation (e.g., the first risk model). The graph may display four distinct survival curves representing different risk groups plotted over time, with survival probability on the y-axis ranging from 0.6 to 1.0 and time in days on the x-axis extending to approximately 3500 days. The curves may represent low risk (less than 2.5%), borderline risk (2.5% to less than 3.75%), intermediate risk (3.75% to less than 10%), and high risk (greater than or equal to 10%) groups. Each curve may include shaded regions indicating confidence intervals surrounding the median survival estimates.

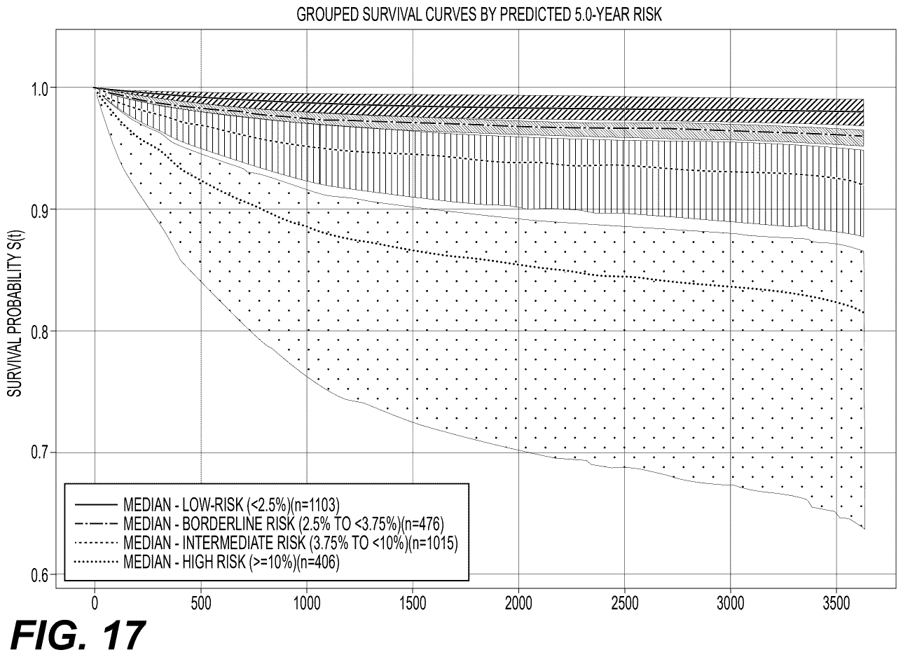

Referring to FIG. 17, grouped survival curves for cardiovascular risk assessment may be presented with four distinct trajectories using a transformer-based lesion scorer (e.g., the second risk model). The graph may display survival probability on the y-axis ranging from 0.6 to 1.0 versus time in days on the x-axis extending to approximately 3500 days. The curves may represent the same risk categories as FIG. 16. Each curve may include shaded regions representing confidence intervals surrounding the median survival estimates.

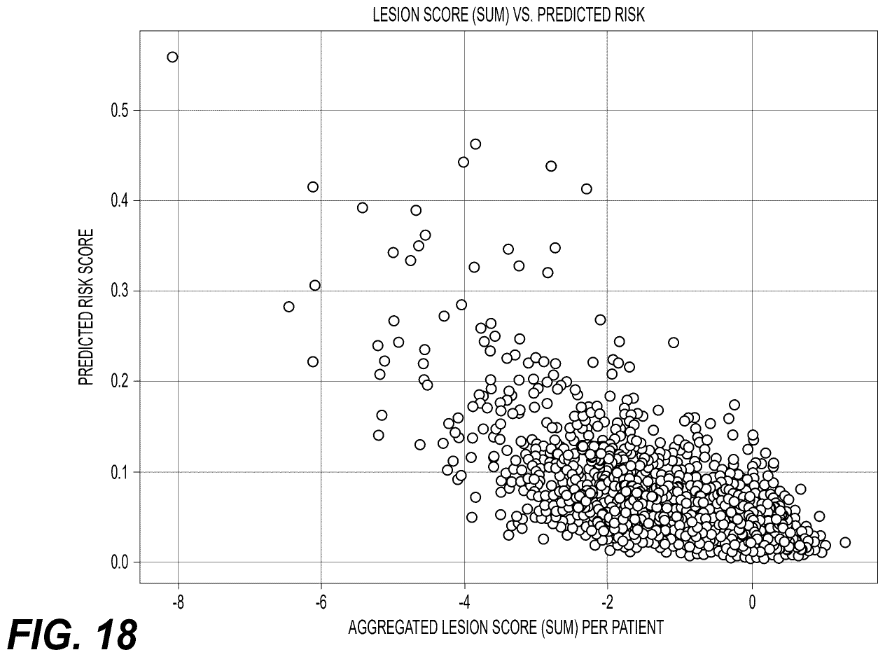

Referring to FIG. 18, a scatter plot may show the relationship between aggregated lesion score and predicted risk across patients for an MLP lesion scorer with simple aggregation (e.g., the first risk model). The x-axis may represent aggregated lesion score (sum) per patient ranging from approximately −8 to 0, while the y-axis may show predicted risk score ranging from 0.0 to approximately 0.55. The plot may contain numerous data points that demonstrate a scattered distribution pattern, with the highest density of points concentrated in the region where lesion scores are closer to 0 and predicted risk scores are below 0.2.

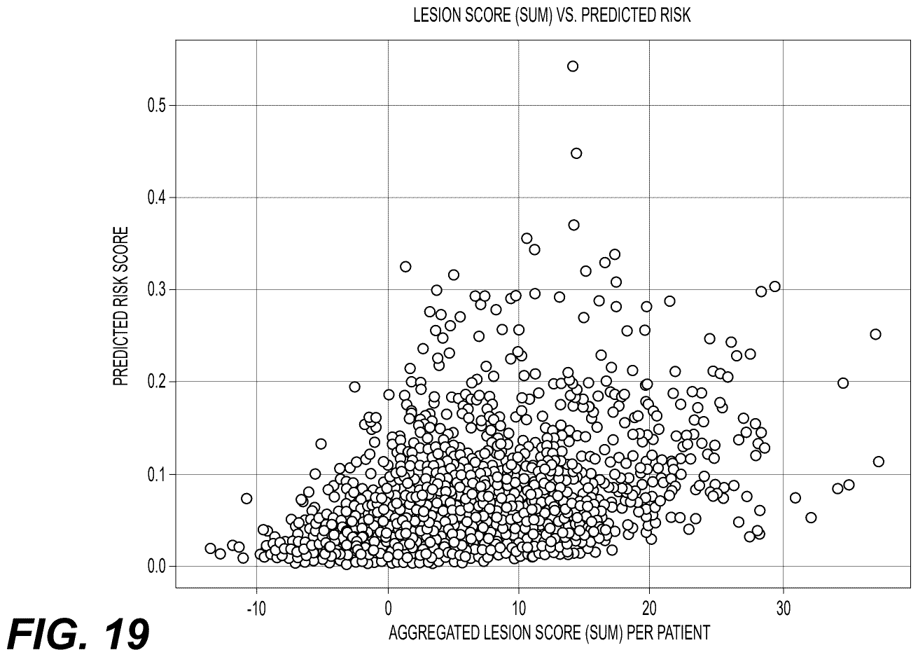

Referring to FIG. 19, a scatter plot may show lesion score versus predicted risk for a transformer-based lesion scorer (e.g., the second risk model). The x-axis may represent aggregated lesion score (sum) per patient ranging from approximately −10 to 30, while the y-axis may show predicted risk score ranging from 0.0 to approximately 0.55. The plot may demonstrate a scattered distribution pattern, with the highest density of points concentrated in the region where lesion scores are between 0 and 10 and predicted risk scores are below 0.2.

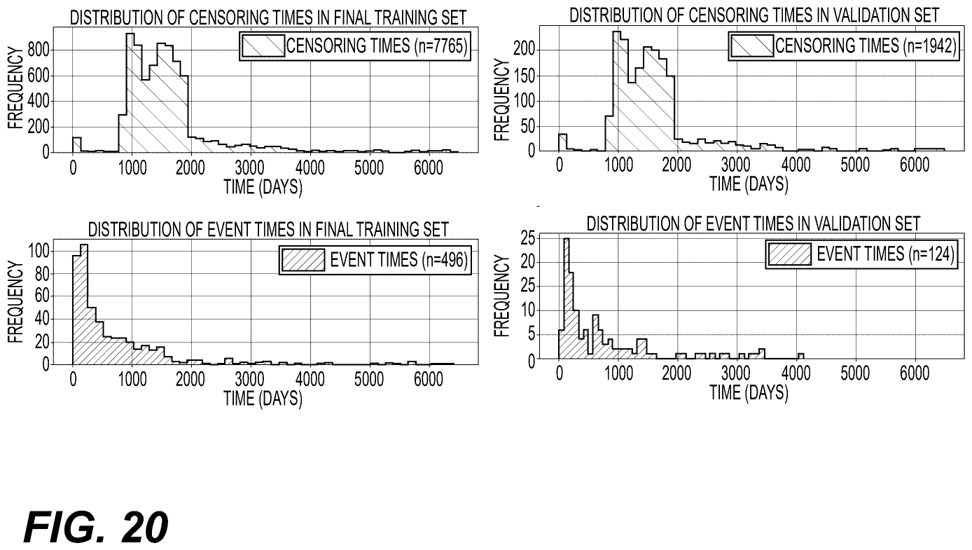

Referring to FIG. 20, distribution plots may show censoring and event times in both training and validation datasets. The upper two graphs may display the distribution of censoring times and event times in the final training set. The lower two graphs may present similar distributions for the validation set. All graphs may share a common x-axis measuring time in days extending to approximately 6000 days. The censoring times distributions in both datasets may demonstrate peaks in the middle time range, while the event times distributions may show higher frequencies at earlier time points with long tails extending toward later times.

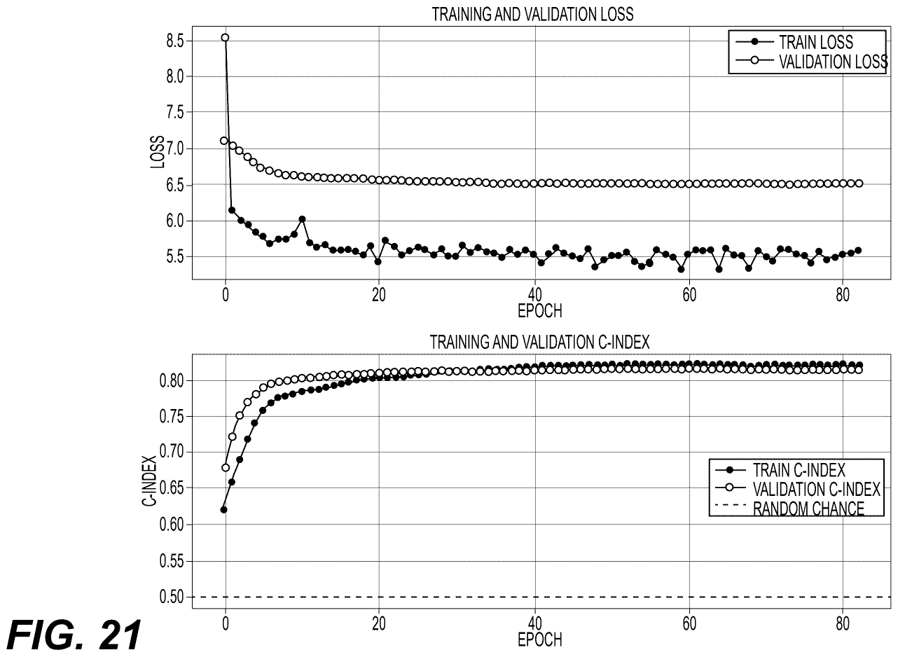

Referring to FIG. 21, training and validation metrics for an MLP lesion scorer using simple aggregation may be displayed over approximately 80 epochs with two vertically arranged graphs. The upper graph may show Training and Validation Loss with the Train Loss curve starting at approximately 8.5 and rapidly decreasing to stabilize around 5.5, and the Validation Loss curve beginning near 7.0 and gradually decreasing to stabilize around 6.5. The lower graph may display Training and Validation C-Index with both curves starting near 0.6 and increasing to plateau around 0.8.

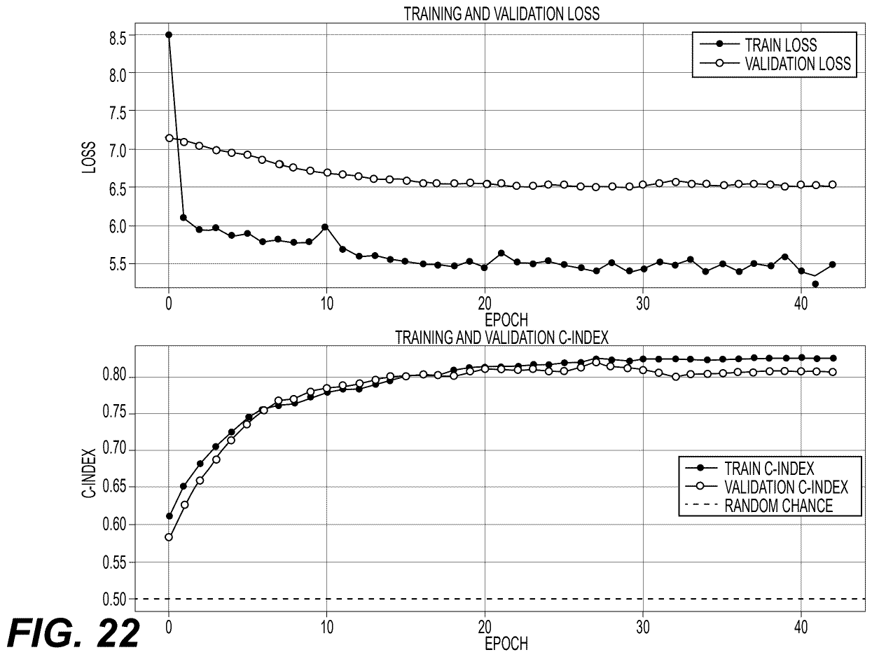

Referring to FIG. 22, training and validation metrics for a transformer-based lesion score model may be shown over approximately 45 epochs. The figure may contain two vertically arranged graphs with the upper graph showing Training and Validation Loss curves and the lower graph displaying Training and Validation C-Index metrics. Both loss curves may start at higher values and decrease during training, while the C-Index curves may show improvement from initial values around 0.6 to plateau near 0.8.

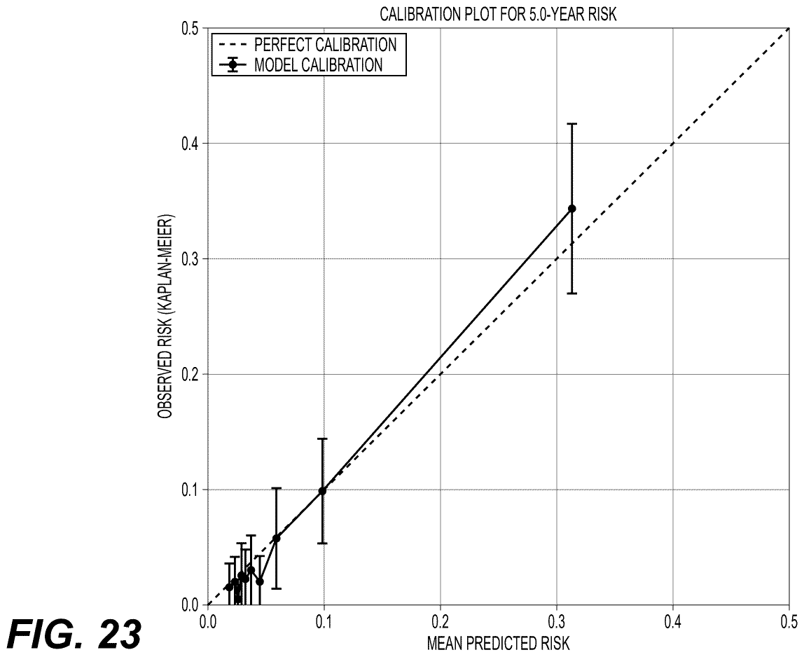

Referring to FIG. 23, a calibration plot for 5.0-year cardiovascular risk assessment may show model reliability for an MLP lesion scorer with simple aggregation (e.g., the first risk model). The plot may display a dashed diagonal line representing perfect calibration and a line with error bars representing model calibration performance. The x-axis may show mean predicted risk ranging from 0.0 to 0.5, while the y-axis may show observed risk (Kaplan-Meier) also ranging from 0.0 to 0.5. The model calibration line may track close to the perfect calibration line at lower risk values below 0.1, with some deviation above the perfect calibration line around 0.2.

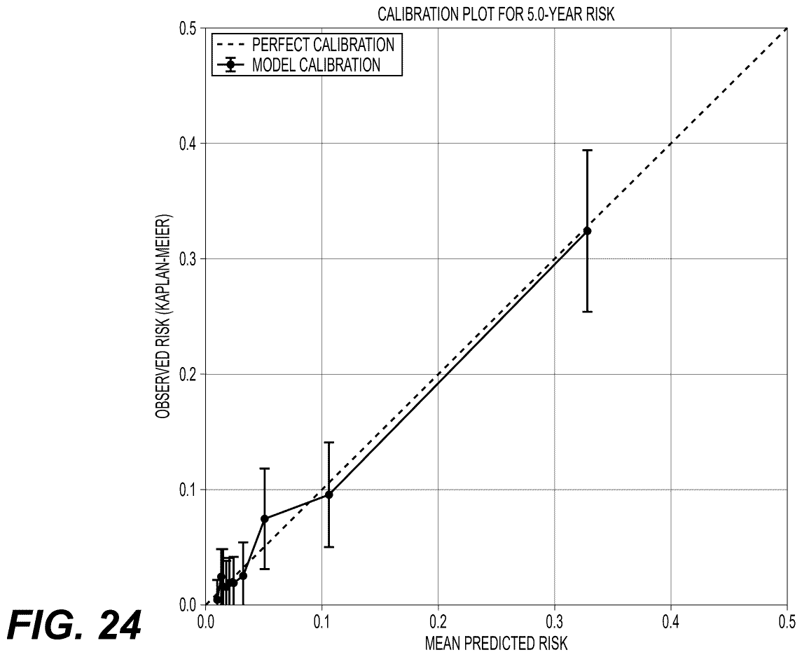

Referring to FIG. 24, another calibration plot may show predicted risk versus observed risk with similar axes to previous calibration plots for a transformer-based lesion scorer (e.g., the second risk model). The model calibration line may track close to the perfect calibration line at lower risk values below 0.1, with some deviation above the perfect calibration line around 0.3. The calibration performance may show reliability across most risk ranges with some deviation at higher levels.

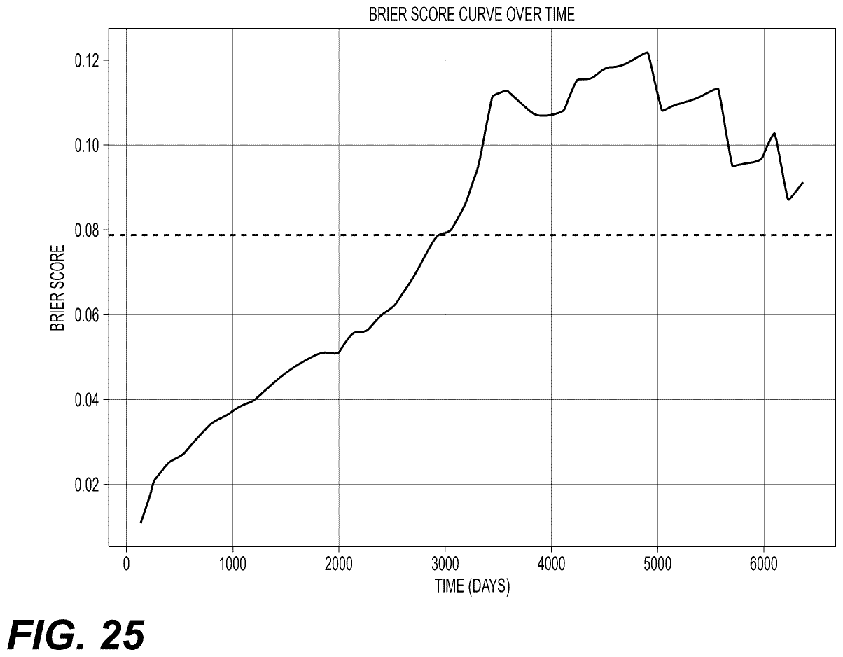

Referring to FIG. 25, a Brier score curve may be plotted over an extended time period of approximately 6000 days for an MLP lesion scorer with simple aggregation (e.g., the first risk model). The graph may display a curve representing Brier score values on the y-axis ranging from 0 to approximately 0.14, with a horizontal dashed reference line positioned at approximately 0.08. The Brier score curve may demonstrate an initial steep increase from near zero, intersecting the reference line around day 3000, and continues to rise more gradually thereafter.

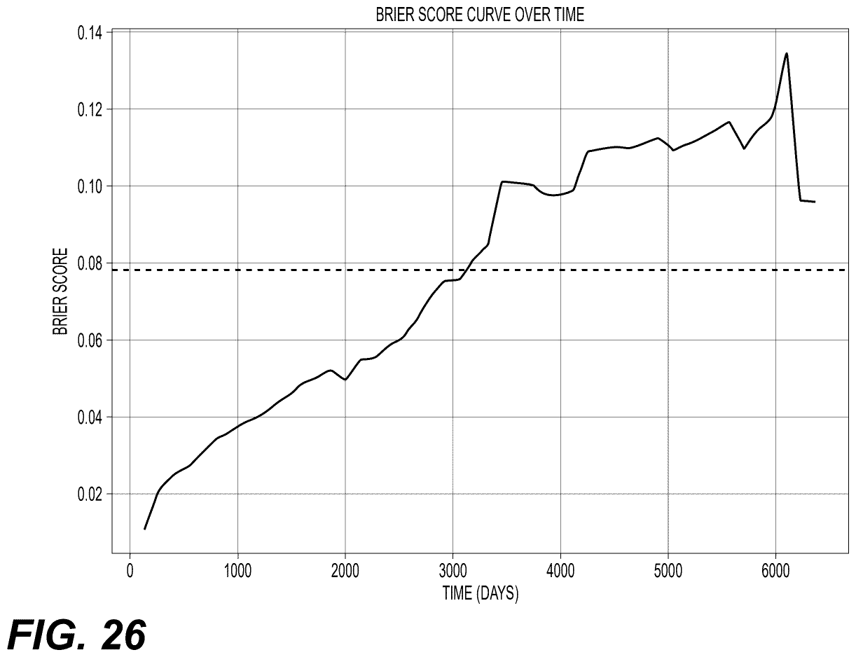

Referring to FIG. 26, Brier score evolution over time may be shown with a similar pattern to FIG. 25, but for a transformer-based lesion scorer (e.g., the second risk model). The graph may display a curve representing Brier score values ranging from 0 to approximately 0.14, with a horizontal dashed reference line at approximately 0.08. The Brier score curve may demonstrate an initial steep increase from near zero, intersecting the reference line around day 3000, and continues to rise more gradually thereafter.

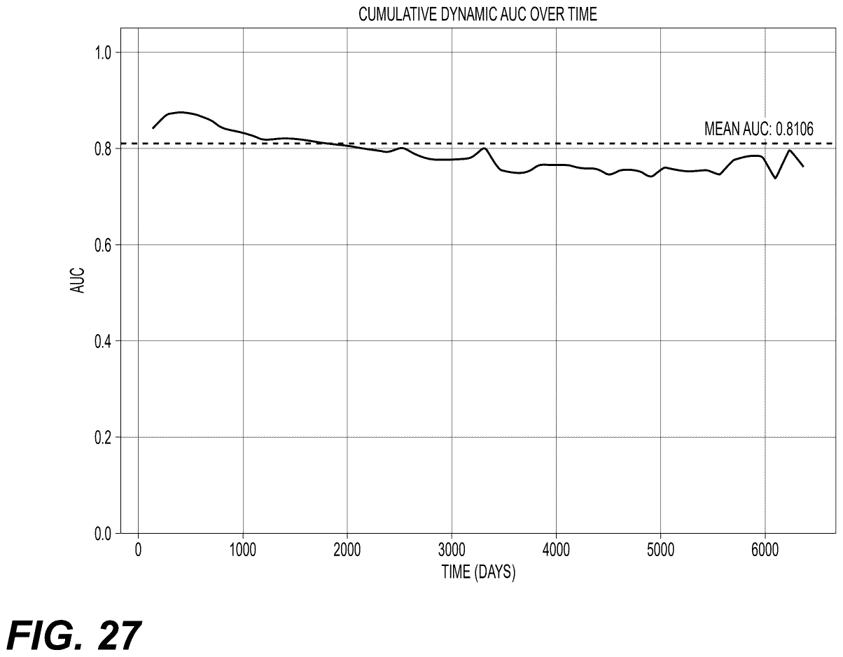

Referring to FIG. 27, cumulative dynamic AUC performance may be depicted over an extended time period of approximately 6000 days for an MLP lesion scorer with simple aggregation (e.g., the first risk model). The graph may display a curve representing dynamic AUC measurements that starts at approximately 0.85 and gradually declines over the measurement period. A horizontal dashed line may indicate the mean AUC value of 0.8106. The AUC curve may demonstrate some fluctuations throughout the measurement period while generally maintaining values above 0.75.

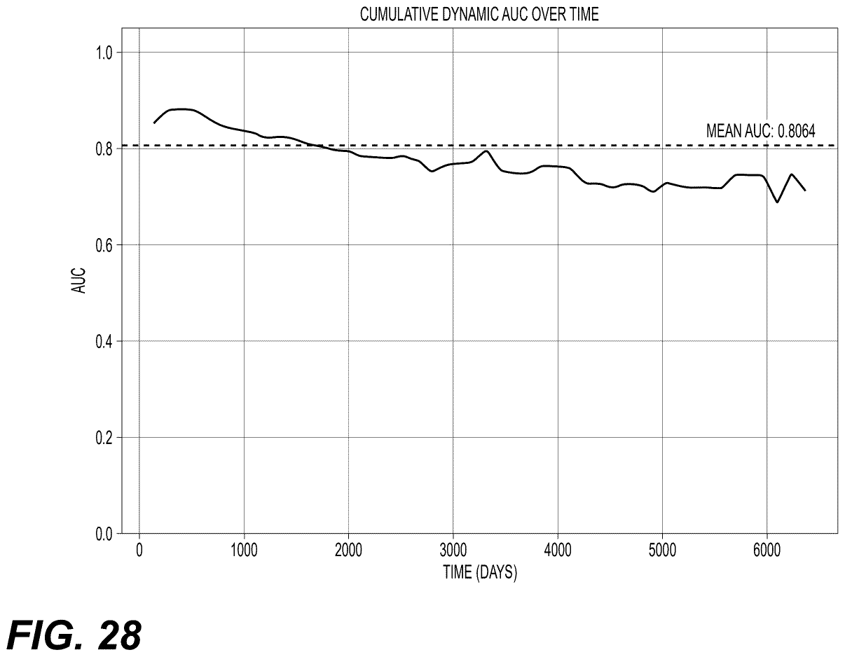

Referring to FIG. 28, cumulative dynamic AUC performance may be presented showing values over approximately 6000 days for a transformer-based lesion scorer (e.g., the second risk model). The graph may display a curve representing dynamic AUC values that starts at approximately 0.85 and shows gradual decline over the measurement period, with a horizontal dashed line indicating a Mean AUC value of 0.8064. The AUC curve may demonstrate relatively stable performance in the early period before showing a gradual decline, maintaining values generally above 0.7 throughout the observation period.

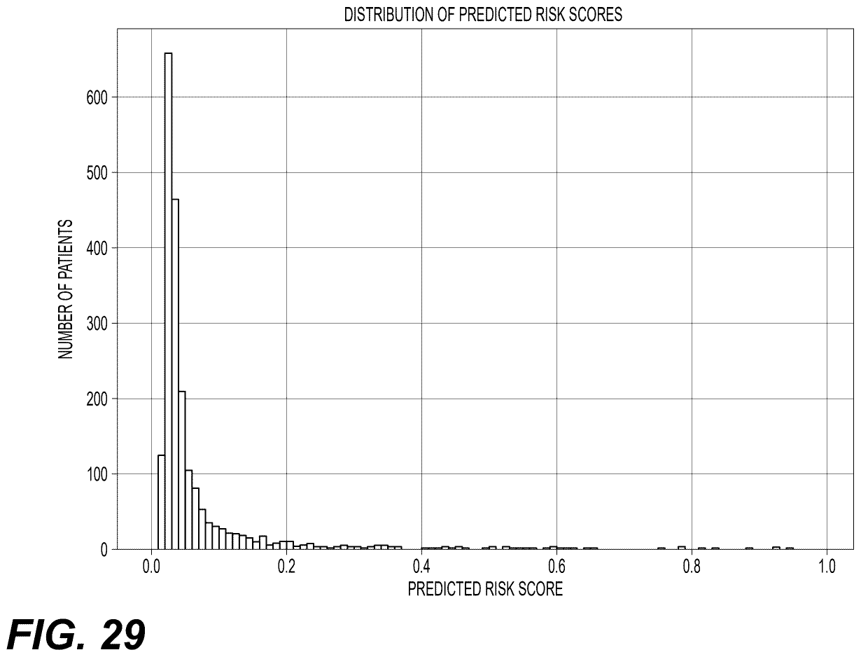

Referring to FIG. 29, a histogram may display the distribution of predicted risk scores across patients for an MLP lesion scorer with simple aggregation (e.g., the first risk model). The x-axis may represent predicted risk scores ranging from 0.0 to 1.0, while the y-axis may show the number of patients ranging from 0 to approximately 650. The distribution may exhibit a heavily right-skewed pattern, with the highest frequency occurring near zero risk scores and rapidly decreasing as risk scores increase.

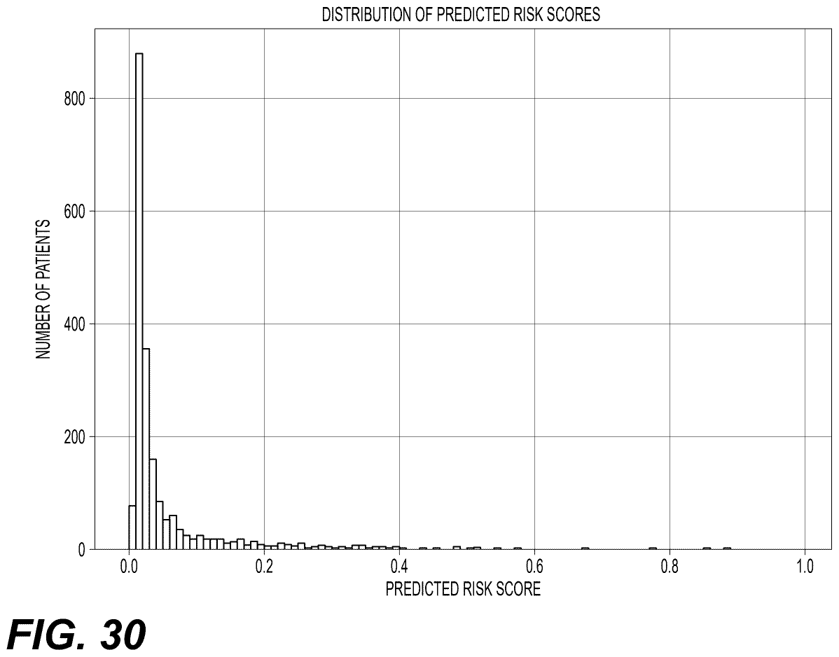

Referring to FIG. 30, predicted risk score distribution may be displayed showing patient numbers versus risk scores for a transformer-based lesion scorer (e.g., the second risk model). The x-axis may represent predicted risk scores ranging from 0.0 to 1.0, while the y-axis may show the number of patients ranging from 0 to approximately 900. The distribution may exhibit a heavily right-skewed pattern, with the highest frequency occurring near zero risk scores and rapidly decreasing as risk scores increase.

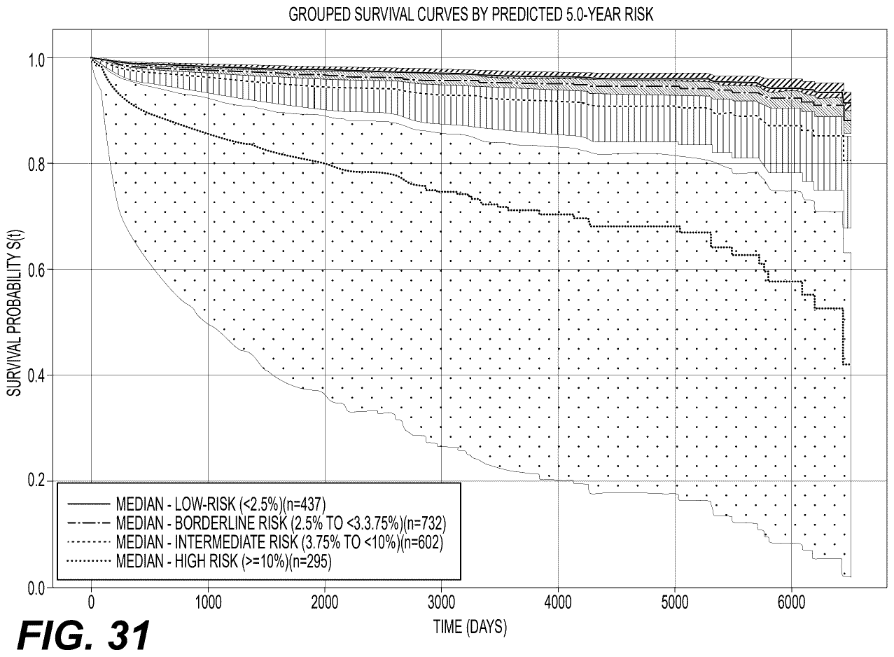

Referring to FIG. 31, grouped survival curves based on predicted 5.0-year cardiovascular risk assessment may be illustrated over an extended follow-up period for an MLP lesion scorer with simple aggregation (e.g., the first risk model). The graph may display four distinct survival curves representing different risk groups plotted over time, with survival probability on the y-axis ranging from 0.0 to 1.0 and time in days on the x-axis extending to approximately 6000 days. Each curve may include shaded regions representing confidence intervals surrounding the median line.

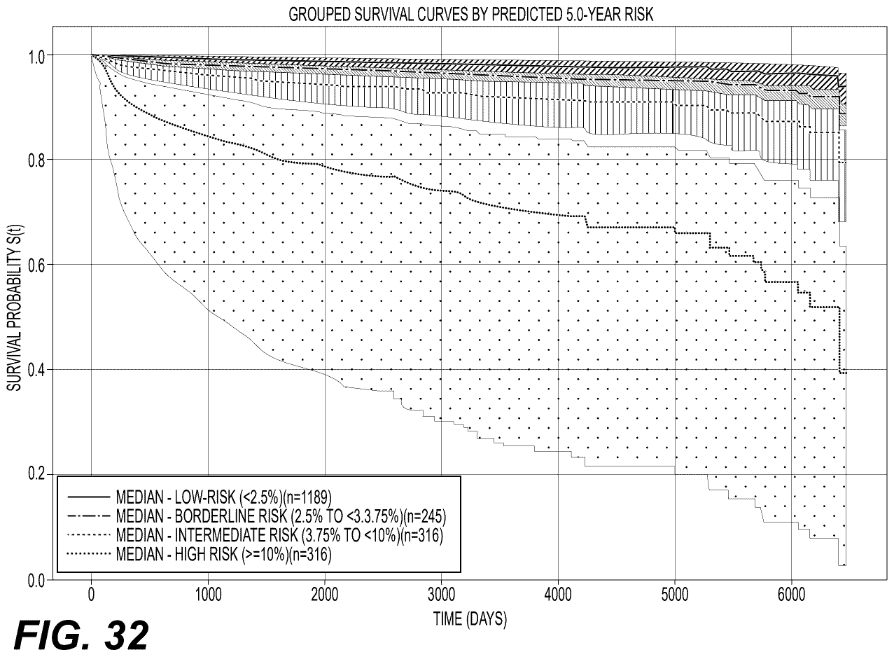

Referring to FIG. 32, grouped survival curves based on predicted 5.0-year risk assessment may be presented over approximately 6,000 days for a transformer-based lesion scorer (e.g., the second risk model). The graph may display four distinct survival curves representing different risk groups with survival probability on the y-axis ranging from 0.0 to 1.0. The curves may represent low-risk (less than 2.5%), borderline risk (2.5% to less than 3.75%), intermediate risk (3.75% to less than 10%), and high risk (greater than or equal to 10%) groups. Each curve may include shaded regions representing confidence intervals surrounding the median line.

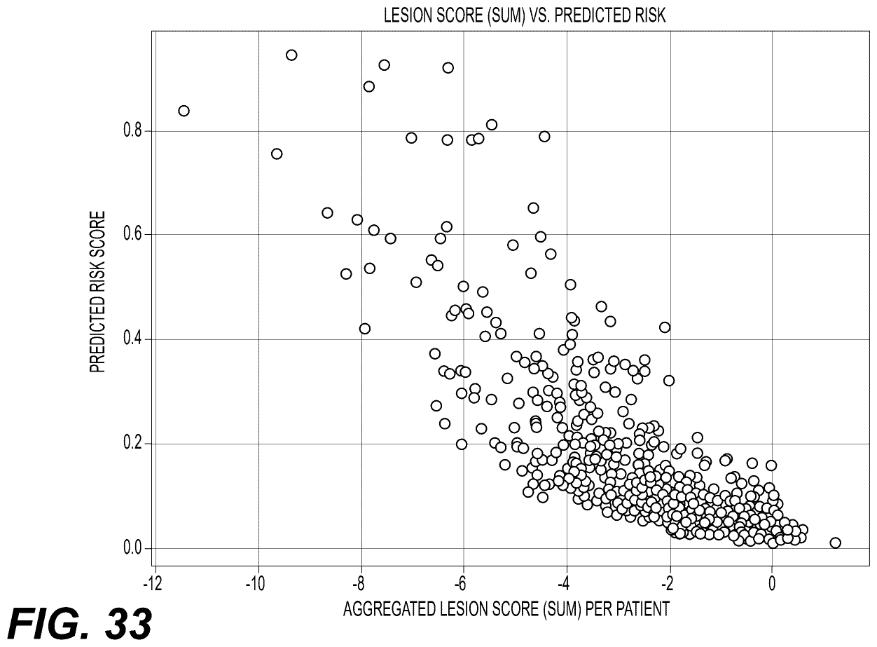

Referring to FIG. 33, a scatter plot may show the relationship between lesion score and predicted risk over a broader range for an MLP lesion scorer with simple aggregation (e.g., the first risk model). The x-axis may represent aggregated lesion score (sum) per patient ranging from approximately −12 to 0, while the y-axis may show predicted risk score ranging from 0.0 to 1.0. The plot may contain numerous data points that demonstrate a scattered distribution pattern, with the highest density of points concentrated in the region where lesion scores are between −2 and 0 and predicted risk scores are below 0.2.

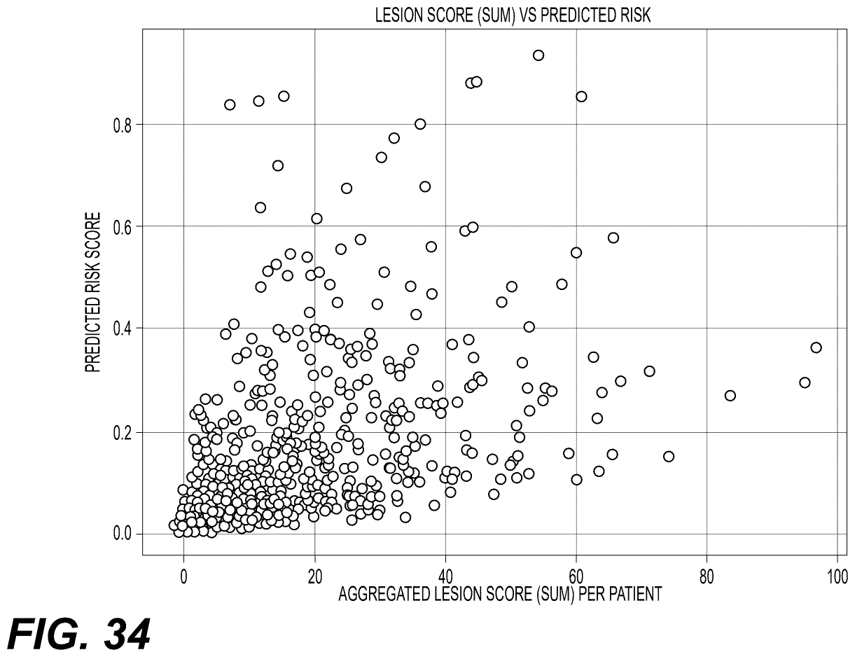

Referring to FIG. 34, a scatter plot may show lesion score versus predicted risk displaying positive correlation patterns for a transformer-based lesion scorer (e.g., the second risk model). The x-axis may represent aggregated lesion score (sum) per patient ranging from 0 to approximately 100, while the y-axis may show predicted risk score ranging from 0.0 to approximately 0.9. The plot may demonstrate a scattered distribution pattern, with the highest density of points concentrated in the region where lesion scores are between 0 and 20 and predicted risk scores are below 0.2.

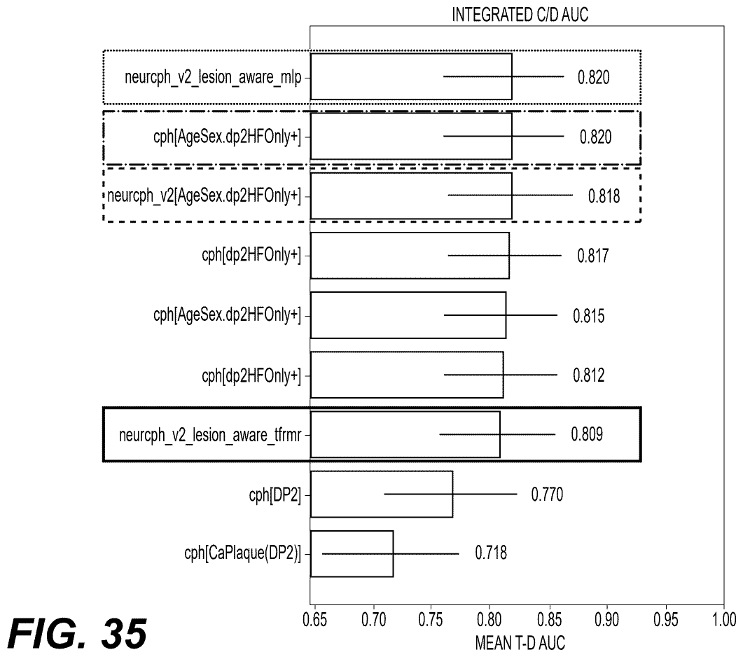

Referring to FIG. 35, integrated C/D AUC performance comparison may be illustrated across different model configurations. The graph may display multiple horizontal bars representing different model approaches, with MLP-based models and baseline Cox regression models achieving high mean T-D AUC values. The comparison may demonstrate the relative performance of different risk model architectures.

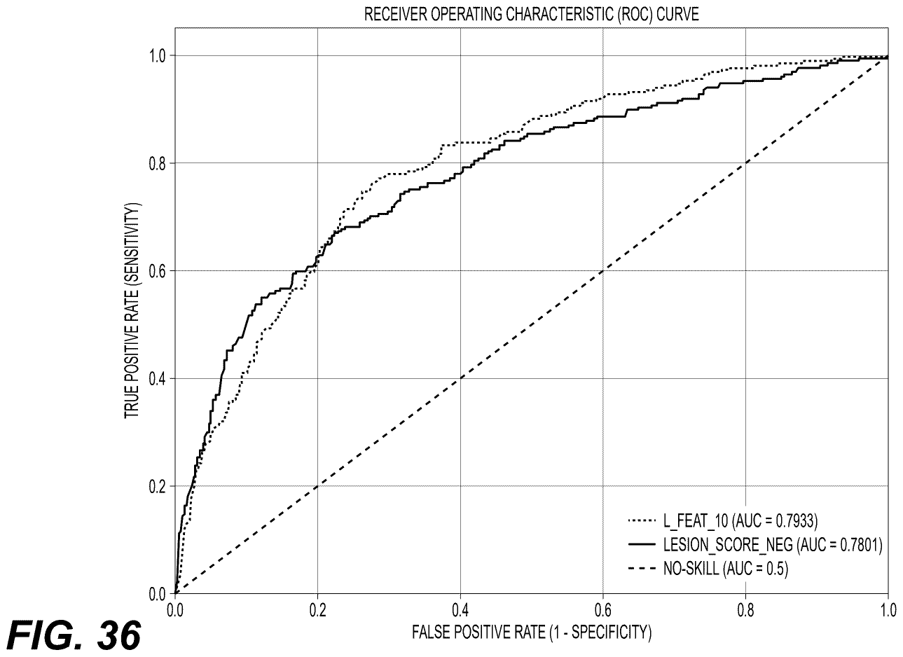

Referring to FIG. 36, a ROC curve may compare model performance across different approaches for an MLP lesion scorer with simple aggregation (e.g., the first risk model). The graph may display curves with different AUC values and a dashed diagonal line labeled “No-Skill” with an AUC value of 0.5. The curves may plot the True Positive Rate (sensitivity) on the y-axis ranging from 0.0 to 1.0 against the False Positive Rate (1-specificity) on the x-axis also ranging from 0.0 to 1.0. The ROC curve may be used for predicting whether a lesion was involved in an acute coronary syndrome event (ACS).

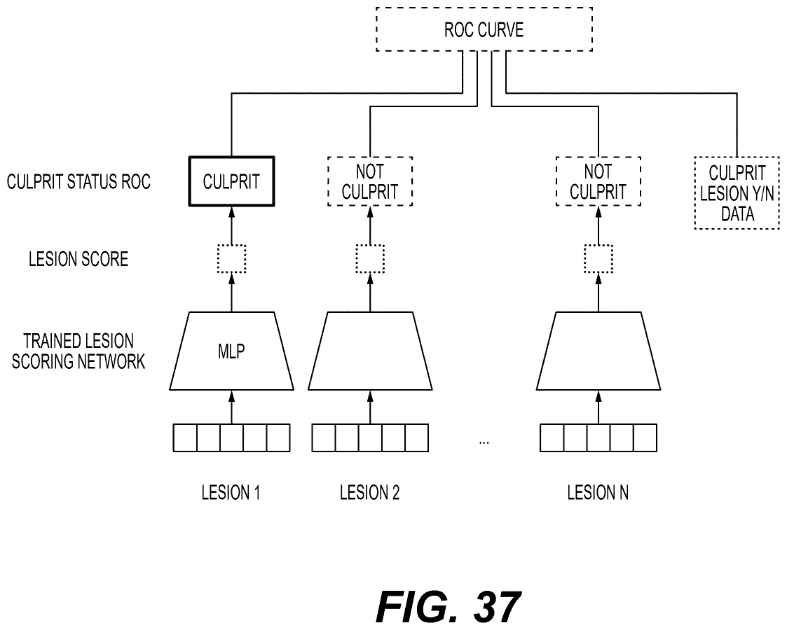

Referring to FIG. 37, a block diagram of a lesion-aware risk prediction system may be presented with MLP lesion scoring and simple aggregation mechanisms. The system may process patient data from multiple sources including various datasets, with multiple parallel processing paths for analyzing individual lesions. The architecture may include separate inputs for first lesion input 1, second lesion input 2, and additional lesions N, indicating the system's capability to handle variable numbers of lesions per patient. Each lesion may be processed through shared lesion scoring networks implemented as multi-layer perceptrons. The individual lesion scores may be aggregated using a summation operation, and the system may combine this aggregated lesion score through concatenation with non-lesion features and non-image content features.

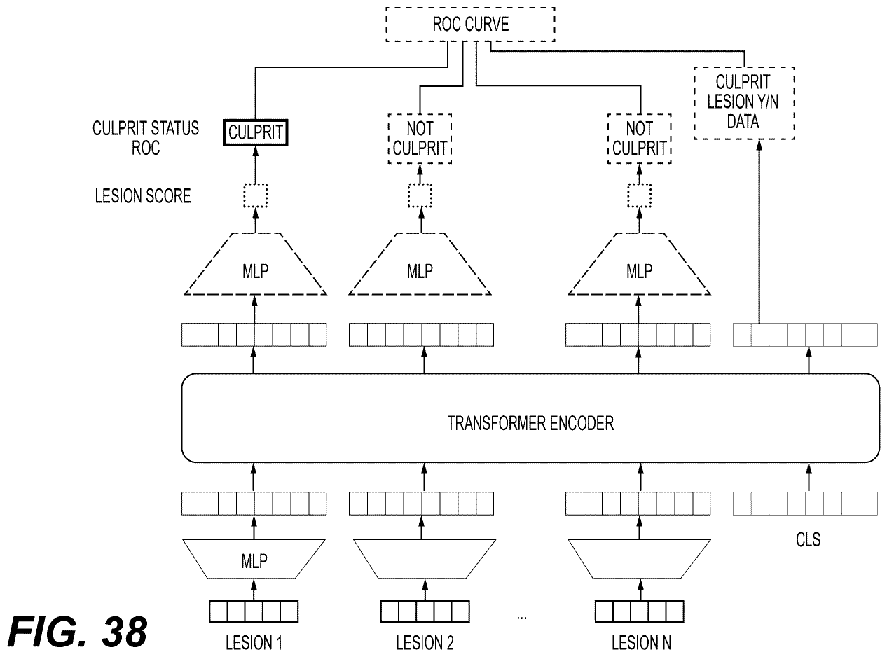

Referring to FIG. 38, a transformer-based architecture for lesion scoring may be illustrated with hierarchical processing structure. The architecture may process multiple lesion inputs through a hierarchical structure, including first lesion input 1 and second lesion input 2, which feed into a Transformer Encoder section that processes the lesion features through MLP layers. The Transformer Encoder may generate contextualized embeddings for each lesion input, enabling the system to capture relationships between different lesions. The architecture may be configured to identify culprit lesion data to generate an ROC curve.

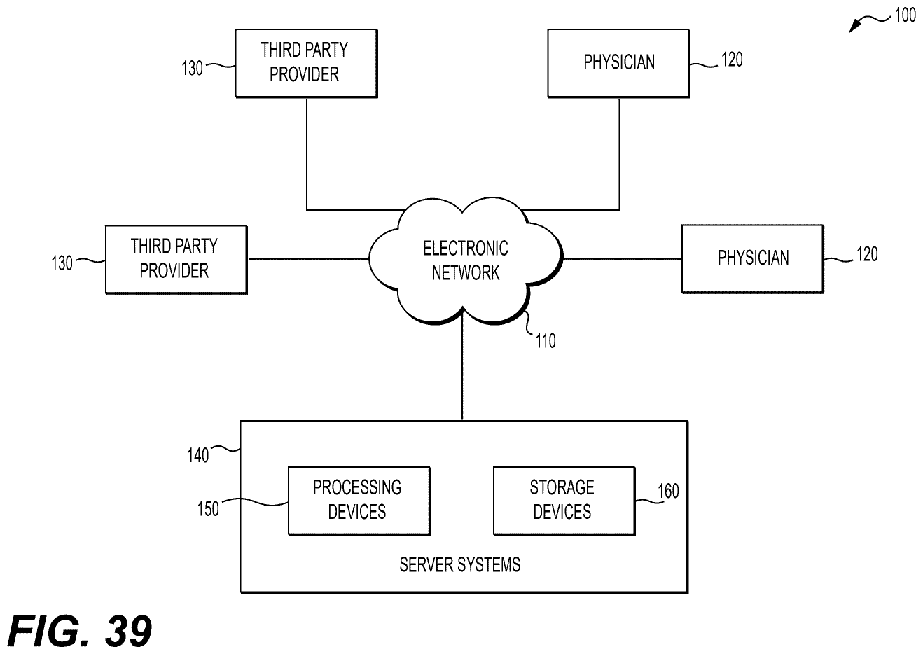

FIG. 39 depicts an example of an environment 100 in which such a computer system may be implemented as server systems 140. In addition to server systems 140, the environment of FIG. 1 further includes a plurality of physicians 120 and third party providers 130, any of which may be connected to an electronic network 110, such as the Internet, through one or more computers, servers, or handheld mobile devices. In FIG. 1, physicians 120 and third party providers 130 may each represent a computer system, as well as an organization that uses such a system. For example, a physician 120 may be a hospital or a computer system of a hospital.

Physicians 120 or third party providers 130 may create or otherwise obtain medical images, such as images of the cardiac, vascular, or organ systems, of one or more patients. Physicians 120 or third party providers 130 may also obtain any combination of patient-specific (or subject-specific) information, such as age, medical history, blood pressure, blood viscosity, and other types of patient-specific information. Physicians 120 or third party providers 130 may transmit the patient-specific information to server systems 140 over the electronic network 110.

Server systems 140 may include one or more storage devices 160 for storing images and data received from physicians 120 or third party providers 130. The storage devices 160 may be considered to be components of the memory of the server systems 140. Server systems 140 may also include one or more processing devices 150 for processing images and data stored in the storage devices and for performing any computer-implementable process described in this disclosure. Each of the processing devices 150 may be a processor or a device that include at least one processor.

In some embodiments, server systems 140 may comprise or utilize a cloud computing platform with scalable resources for computations or data storage, and may run an application for performing methods described in this disclosure on the cloud computing platform. In such embodiments, any outputs may be transmitted to another computer system, such as a personal computer, for display or storage.

Other examples of computer systems for performing methods of this disclosure include desktop computers, laptop computers, and mobile computing devices such as tablets and smartphones.

A computer system, such as server systems 140, may include one or more computing devices. If the one or more processors of the computer system is implemented as a plurality of processors, the plurality of processors may be included in a single computing device or distribute among a plurality of computing devices. If a computer system comprises a plurality of computing devices, the memory of the computer system may include the respective memory of each computing device of the plurality of computing devices.

In some aspects, an end-to-end learning approach may be used for EAT and PCAT characterization. Instead of relying on pre-defined features, the method may directly learn the relationship between the raw volumetric image intensities of the EAT and PCAT and user-determined endpoints. This approach may utilize deep learning models, such as convolutional neural networks (CNNs), which are capable of automatically learning hierarchical representations from the raw image data. By learning directly from the raw data, the method may have the potential to capture subtle patterns and relationships that may be missed by hand-crafted features. As used herein, “raw” medical image data refers to image-related data acquired from one or more medical imaging systems and includes, without limitation: (i) scanner measurement data prior to reconstruction (e.g., projection data, sinograms, k-space data, list-mode data, radiofrequency data, or other modality-specific measurement representations), (ii) reconstructed image data (e.g., voxel or pixel data forming one or more 2D images, 3D volumes, or 4D time series) prior to segmentation, feature extraction, or lesion-level quantification, and/or (iii) minimally processed versions of the foregoing (e.g., resampled, denoised, normalized, motion-corrected, or artifact-reduced) that preserve diagnostically meaningful signal content. “Raw” does not necessarily require that the data be uncompressed, unformatted, or unmodified; rather, “raw” distinguishes image-domain or measurement-domain data from higher-level derived outputs such as segmented masks, engineered feature tables, or clinician-authored interpretations. “Raw” may encompasses subsets of such data used for analysis, such as multi-planar reformats, curved planar reformats, and image patches or crops centered on candidate or identified lesions. In this context, a patch/crop is still “raw image data” when it is a direct excerpt of the acquired/reconstructed image values (optionally after non-semantic preprocessing such as resampling or normalization) rather than a derived semantic representation (e.g., a segmentation mask or engineered feature vector).

In some aspects, training for EAT/PCAT characterization may include collecting a set of CCTA scans with corresponding desired outputs of the model. The desired outputs may be one or more values for the entire image and/or one or more values for one or more regions in the image. The CCTA scan may be processed and the EAT and/or PCAT region or subregion may be extracted. This may be done through manual, semi-automated or automated segmentation, potentially combined with thresholding of the intensities at pre-defined thresholds. A deep learning model may be trained that takes the HUs from EAT and/or PCAT regions as input and predicts the corresponding desired outputs.

In some aspects, inference/usage for EAT/PCAT characterization may include processing a CCTA scan and extracting the EAT and/or PCAT region or subregion. The HUs from the EAT and/or PCAT regions may be provided to the deep learning model and the output may be extracted for a desired task.

In some aspects, local plaque progression may be predicted with local PCAT values. Training may include collecting a set of longitudinal CCTA data, i.e., pairs of CCTA data acquired over time (baseline and follow-up scans). From each pair of CCTA scans, the coronary trees, the outer walls, and the amount of plaque at each point of the coronary artery trees may be extracted. Machine learning and deep learning methods may be combined with human review to obtain outer wall segmentations and plaque quantifications. The PCAT voxels in the baseline scan may be defined as all voxels outside the outer wall and within a predefined distance (e.g., 3 mm) of it. The two coronary artery trees may be co-registered and a correspondence between points on the coronary artery tree in the baseline scan and points in the follow-up scan may be established. For each point in the baseline scan, the difference in local plaque volume between the baseline and follow-up scans may be determined. A CNN may be trained that takes, as input for each centerline point, the local PCAT voxels from the baseline scan, the time between the scans and outputs the plaque difference.

In some aspects, inference/usage for predicting local plaque progression may include receiving a CCTA scan and extracting from the CCTA scans, the coronary trees, the outer walls, and the amount of plaque at each point of the coronary artery trees. The PCAT voxels in the scan may be defined. The trained CNN may be provided a time to a point in the future and, for each centerline point, the local PCAT voxels. The CNN may then output for each centerline point the predicted plaque progression based on the local PCAT voxels.

In some aspects, enhanced outputs may be provided. Risk of Acute Coronary Syndrome (ACS) may be predicted: instead of just predicting plaque volume changes, the model may be trained to directly predict the risk of ACS within a specific timeframe (e.g., 1, 3, or 5 years). This may be a binary classification (high/low risk) or a probability score. Plaque composition changes may be predicted: changes in plaque composition (e.g., calcified, non-calcified, fibrous) may be predicted in addition to volume changes. Vulnerable plaque features may be predicted: specific features associated with vulnerable plaques, such as thin-cap fibrous cap (TCFC), large necrotic core, or spotty calcifications may be predicted. Hemodynamic properties may be estimated: changes in blood flow and wall shear stress based on the predicted plaque progression may be estimated. Medication suggestions may be provided: based on the volumes present, intensity of HU values, and other input variables, more or specific medications may be suggested.

In some aspects, different deep learning architectures may be used. Transformers, particularly Vision Transformers (ViT), may be used and have shown promise in medical image analysis. Such transformers can capture long-range dependencies in the image data, potentially improving the prediction of plaque progression. Graph Neural Networks (GNNs) may be used and are well-suited for analyzing data with underlying graph structures. The coronary artery tree can be represented as a graph, where nodes are centerline points and edges represent connections between them. Multi-task learning may be used to train a single model to predict multiple outputs simultaneously (e.g., plaque volume change, plaque composition change, risk of ACS).

In some aspects, improved PCAT definitions may be used. Different distances may be used instead of a fixed 3 mm distance, such as 1 mm, 2 mm, 4 mm, 5 mm, 6 mm, etc. An adaptive PCAT definition may be used that takes into account the local vessel size and plaque burden.

In some aspects, additional inputs may be utilized. CT acquisition parameters may be utilized as inputs: due to the dependence of PCAT attenuation on kVp, it may be beneficial to provide kVp and potentially other CT acquisition parameters, such as the reconstruction algorithm, to the model. Patient characteristics may be utilized: plaque progression and other outputs can potentially be better predicted if patient demographics and potentially patient risk factors (hyperlipidemia, diabetes status, etc.) are considered as inputs to the model.

In some aspects, inference/usage enhancements may be provided. Uncertainty quantification may provide uncertainty estimates along with the predictions. Visualization tools may be developed to visualize the predicted plaque progression and other outputs on the CCTA scan. Explainability methods may be explored to make the model's predictions more interpretable.

In some aspects, alternative input image representations may be used. A masked 3D image volume may provide a 3D image volume with a mask of the EAT and/or PCAT voxels, providing a sparse representation of the native 3D geometry of the heart and coronary arterial tree. A masked straightened curvilinear planar reformation (sCPR) may use a sCPR representation and mask out voxels that do not belong to the PCAT and/or EAT. An unrolled masked sCPR may involve taking the masked sCPR and reformatting the image such that instead of the voxels of interest being represented in a ring with the inner and outer regions of that ring masked out, the ring is flattened and the masked voxels are removed from the representation. A centerline tree representation may be conducive to use with GNNs, where inputs at each centerline location represent the spatially contiguous intensities of PCAT in the voxels of the corresponding vessel cross-section.

In some aspects, additional inputs may include patient risk factors or history: EAT or PCAT volumes may be related to various aspects of a patient's history including medications taken, age, gender, present/past medical history, and/or family history. Other medical image data (IVUS, OCT, MRI, etc.) may provide more information about the EAT or PCAT volumes in the patient. Raw image data prior to reconstruction may provide signals that could provide more information in identifying EAT or PCAT volumes.

A number of implementations have been described. Nevertheless, it will be understood that various modifications may be made without departing from the spirit and scope of the disclosure. Accordingly, other implementations are within the scope of the following claims.

1. A method of performing a cardiovascular risk assessment for a patient, comprising:
extracting a plurality of cardiovascular plaque, geometric, and functional significance parameters from raw patient-specific medical image data;
analyzing the plurality of cardiovascular plaque, geometric, and functional significance parameters using a risk score model to output patient-specific risk values, wherein the risk score model is configured to correlate the plurality of cardiovascular plaque, geometric, and functional significance parameters with predicted cardiovascular outcomes; and
determining a likelihood of a major adverse cardiovascular event for the patient based on the patient-specific risk values.

2. The method of claim 1, wherein the raw patient-specific medical image data comprises coronary computed tomography angiography data.

3. The method of claim 1, wherein extracting the plurality of cardiovascular plaque, geometric, and functional significance parameters comprises identifying individual coronary lesions from the raw patient-specific medical image data and extracting lesion-specific features for each identified lesion.

4. The method of claim 3, wherein the plurality of cardiovascular plaque, geometric, and functional significance parameters correlate to lesion-specific features.

5. The method of claim 1, wherein the plurality of cardiovascular plaque, geometric, and functional significance parameters comprise calcified plaque volume, non-calcified plaque volume, and plaque burden measurements.

6. The method of claim 1, wherein the plurality of cardiovascular geometric parameters comprise diameter stenosis measurements, vessel curvature measurements, and positive remodeling indices.

7. The method of claim 1, wherein the risk score model comprises a neural network architecture including a lesion scoring network configured to generate individual lesion scores for each detected lesion.

8. The method of claim 7, further comprising aggregating the individual lesion scores to generate an aggregated lesion score by summing the individual lesions scores.

9. The method of claim 8, wherein aggregating the individual lesion scores comprises using a transformer encoder to generate contextualized embeddings for lesion aggregation.

10. The method of claim 8, further comprising combining the aggregated lesion score with non-lesion cardiovascular features extracted from the raw patient-specific medical image data to generate combined feature data.

11. The method of claim 10, wherein the non-lesion cardiovascular features comprise epicardial adipose tissue volume, epicardial adipose tissue burden, left ventricular myocardial mass, pericoronary adipose tissue measurements, features derived from cardiac ventricular and atrial volumes and left atrial appendage morphology, aortic plaque, carotid plaque, and cerebrovascular plaque.

12. The method of claim 11, further comprising processing the combined feature data through a risk prediction neural network to generate a cardiovascular risk score indicating probability of major adverse cardiac events.

13. The method of claim 12, wherein the risk prediction neural network is trained with loss functions associated with time-to-event modeling.

14. The method of claim 13, wherein the risk prediction neural network implements survival modeling for time-to-event analysis.

15. The method of claim 1, further comprising generating risk stratification categories based on the patient-specific risk values, wherein the risk stratification categories classify the patient into discrete risk groups for major adverse cardiovascular events.

16. The method of claim 1, wherein the raw patient-specific medical image data further comprises magnetic resonance imaging data, echocardiography data, or nuclear imaging data.

17. The method of claim 4, wherein the functional significance parameters comprise coronary flow reserve measurements, myocardial perfusion indices, or wall motion abnormality scores.

18. The method of claim 1, further comprising validating the risk score model using cross-validation techniques and generating performance metrics including area under the curve, sensitivity, and specificity values.

19. The method of claim 1, wherein determining the likelihood of a major adverse cardiovascular event comprises generating time-specific risk predictions for multiple time intervals including one-year, three-year, and five-year risk assessments.

20. A system of determining a cardiovascular risk assessment for a patient, comprising:
a data storage device storing instructions for determining cardiovascular risk; and
a processor configured to execute the instructions to perform operations comprising:
extracting a plurality of cardiovascular plaque, geometric, and functional significance parameters from raw patient-specific medical image data;
analyzing the plurality of cardiovascular plaque, geometric, and functional significance parameters using a risk score model to output patient-specific risk values, wherein the risk score model is configured to correlate the plurality of cardiovascular plaque, geometric, and functional significance parameters with predicted cardiovascular outcomes; and
determining a likelihood of a major adverse cardiovascular event for the patient based on the patient-specific risk values.
